(19) United States
(12) Patent Application Publication
(10) Pub. No.: US 2003/0059856 A1
(43) Pub. Date: Mar. 27, 2003

(54) Methods of screening for agonists and agonists of the interaction between the AXOR8 and AXOR52 receptors and ligands thereof

(76) Inventors: Robert S. Ames JR., Haverford, PA (US); Henry M. Sarau, Harleysville, PA (US); J. Randall Slemmon, Glenview, IL (US); Dean E. McNulty, Philadelphia, PA (US); Lisa Vawter, Coopersburg, PA (US); James J. Foley, Radnor, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/132,812

(22) Filed: Apr. 25, 2002

Related U.S. Application Data
(60) Provisional application No. 60/286,234, filed on Apr. 25, 2001.

(51) Int. Cl. G01N 33/567

(52) U.S. Cl. 435/7.21

(57) ABSTRACT

Disclosed are methods for discovering agonists and antagonists of the interaction between monkey AXOR8, human AXOR8, and human AXOR52 receptors with their natural ligands: human BV8-a, mouse BV8-a, frog BV8, human BV8-b, human PRO1186, human PRO1186 variant, and mamba intestinal toxin (herein “MIT”). Such agonists or antagonists can be used in the treatment of several human diseases and disorders, including, but not limited to: bacterial, fungal, protozoan and viral infections, particularly infections caused by HIV-1 or HIV-2; pain; cancers; diabetes, obesity; anorexia; bulimia; asthma; Parkinson's disease; acute heart failure; hypotension; hypertension; urinary retention; osteoporosis; angina pectoris; myocardial infarction; stroke; ulcers; asthma; allergies; benign prostatic hypertrophy; migraine; vomiting; psychotic and neurological disorders, including anxiety, schizophrenia, manic depression, depression, delirium, dementia, and severe mental retardation; and dyskinesias, such as Huntington's disease or Gilles dela Tourett's syndrome.

### CROSS-REFERENCE TO RELATED APPLICATIONS

This application claims benefit to the earlier provisional U.S. Application No. 60/286,234, filed on Apr. 25, 2001, the contents of which are incorporated herein by reference in their entirety.

### FIELD OF THE INVENTION

This invention relates to methods for discovering agonists and antagonists of the interaction between the monkey AXOR8, human AXOR8, and human AXOR52 receptors and their natural ligands. The invention also relates to the use of the identified agonists, antagonists and/or inhibitors, which are potentially useful in the treatment of human diseases/disorders, including, but not limited to: infections such as bacterial, fungal, protozoan and viral infections, particularly infections such as bacterial, fungal, protozoan and viral infections, particularly infections caused by HIV-1 or HIV-2; pain; cancers; diabetes, obesity; anorexia; bulimia; asthma; Parkinson's disease; acute heart failure; hypotension; hypertension; urinary retention; osteoporosis; angina pectoris; myocardial infarction; stroke; ulcers; asthma; allergies; benign prostatic hypertrophy; migraine; vomiting; psychotic and neurological disorders, including anxiety, schizophrenia, manic depression, depression, delirium, dementia, and severe mental retardation; and dyskinesias, such as Huntington's disease or Gilles dela Tourett's syndrome.

### BACKGROUND OF THE INVENTION

It is well established that many medically significant biological processes are mediated by proteins participating in signal transduction pathways that involve G-proteins and/or second messengers, e.g., cAMP (Lefkowitz, Nature, 1991, 351:353-354). Herein these proteins are referred to as proteins participating in pathways with G-proteins or PPG proteins. Some examples of these proteins include the G-protein coupled (GPC) receptors, such as those for adrenergic agents and dopamine (Kobilka, B. K., et al., Proc. Natl Acad Sci., USA, 1987, 84:46-50; Kobilka, B. K., et al., Science, 1987, 238:650-656; Bunzow, J. R., et al, Nature, 1988, 336:783-787), G-proteins themselves, effector proteins, e.g., phospholipase C, adenyl cyclase, and phosphodiesterase, and actuator proteins, e.g., protein kinase A and protein kinase C (Simon, M. I., et al., Science, 1991, 252:802-8).

For example, in one form of signal transduction, the effect of hormone binding is activation of the enzyme, adenylate cyclase, inside the cell. Enzyme activation by hormones is dependent on the presence of the nucleotide GTP. GTP also influences hormone binding. A G-protein connects the hormone receptor to adenylate cyclase. G-protein was shown to exchange GTP for bound GDP when activated by a hormone receptor. The GTP-carrying form then binds to activated adenylate cyclase. Hydrolysis of GTP to GDP, catalyzed by the G-protein itself, returns the G-protein to its basal, inactive form. Thus, the G-protein serves a dual role, as an intermediate that relays the signal from receptor to effector, and as a clock that controls the duration of the signal.

The membrane protein gene superfamily of G-protein coupled receptors has been characterized as having seven putative transmembrane domains. The domains are believed to represent transmembrane a-helices connected by extracellular or cytoplasmic loops. G-protein coupled receptors include a wide range of biologically active receptors, such as hormone, viral, growth factor and neuroreceptors.

G-protein coupled receptors (otherwise known as 7TM receptors) have been characterized as including these seven conserved hydrophobic stretches of about 20 to 30 amino acids, connecting at least eight divergent hydrophilic loops. The G-protein family of coupled receptors includes dopamine receptors which bind to neuroleptic drugs used for treating psychotic and neurological disorders. Other examples of members of this family include, but are not limited to, calcitonin, adrenergic, endothelin, cAMP, adenosine, muscarinic, acetylcholine, serotonin, histamine, thrombin, kinin, follicle stimulating hormone, opsins, endothelial differentiation gene-1, rhodopsins, odorant, and cytomegalovirus receptors.

Most G-protein coupled receptors have single conserved cysteine residues in each of the first two extracellular loops which form disulfide bonds that are believed to stabilize functional protein structure. The 7 transmembrane regions are designated as TM1, TM2, TM3, TM4, TM5, TM6, and TM7. TM3 has been implicated in signal transduction. Phosphorylation and lipidation (palmitylation or farnesylation) of cysteine residues can influence signal transduction of some G-protein coupled receptors. Most G-protein coupled receptors contain potential phosphorylation sites within the third cytoplasmic loop and/or the carboxy terminus. For several G-protein coupled receptors, such as the β-adrenoreceptor, phosphorylation by protein kinase A and/or specific receptor kinases mediates receptor desensitization.

For some receptors, the ligand binding sites of G-protein coupled receptors are believed to comprise hydrophilic sockets formed by several G-protein coupled receptor transmembrane domains, said socket being surrounded by hydrophobic residues of the G-protein coupled receptors. The hydrophilic side of each G-protein coupled receptor transmembrane helix is postulated to face inward and form polar ligand binding site. TM3 has been implicated in several G-protein coupled receptors as having a ligand binding site, such as the TM3 aspartate residue. TM5 serines, a TM6 asparagine and TM6 or TM7 phenylalanines or tyrosines are also implicated in ligand binding.

G-protein coupled receptors can be intracellularly coupled by heterotrimeric G-proteins to various intracellular enzymes, ion channels and transporters (see Johnson, et al., Endoc. Rev., 1989, 10:317-331) Different G-protein α-subunits preferentially stimulate particular effectors to modulate various biological functions in a cell. Phosphorylation of cytoplasmic residues of G-protein coupled receptors have been identified as an important mechanism for the regulation of G-protein coupling of some G-protein coupled receptors. G-protein coupled receptors are found in numerous sites within a mammalian host.

Over the past 15 years, nearly 350 therapeutic agents targeting 7 transmembrane (7 TM) receptors have been successfully introduced onto the market.

Bv8 is a small protein isolated from frog skin which contains 5 disulfide bonds and a signal secretion sequence. Mollay, et al., Eur. J. Pharm. 374: 189-196 (1999). It is homologous to the Mamba Intestinal Toxin-1 protein from snake venom (MIT-1, protein A). Joubert, et al., Physiol. Chem. Bd., 361, S.: 1784-1794 (1980}; Schweitz, et al., FEBS Letters 461: 183-188 (1999). Bv8 and MIT-1 share a similar pattern containing 10 cysteines, indicating that these proteins are members of a larger family. MIT-1 protein from snake venom was the first of these to be purified. Joubert, et al., supra. The frog (Mollay, et al., supra.), mouse (Wechselberger, et al., FEBS Letters, 462: 177-181 (1999)) and human (Li, et al., Mol. Pharm. 59: 692-698 (2001)). Bv8 precursor proteins have since been cloned and expressed, which allowed for the discovery that Bv8 is a bioactive protein ligand. Earlier studies suggest these proteins may be regulators of GI functions. Frog Bv8 stimulated the contraction of guinea-pig ileum (Mollay, et al., supra.), while the prokineticins (human Bv8 proteins) were potent agents for contracting gastrointestinal smooth muscle (Li, et al., supra.). Human PRO1186 was also identified as an angiogenic mitogen which induced proliferation, migration and fenestration of capillary endothelial cells derived from endocrine gland. It was thus named endocrine-gland-derived vascular endothelial growth factor (EG-VEGF). LeCouter, et al., Nature, 412: 877-884 (2001). It may act as a tissue-specific mitogen which regulate proliferation and differentiation of vascular endothelium.

The widely spread expression of messenger RNA for AXOR8, AXOR52 and their human ligands in the central nervous system also indicate these receptors may play a role in the regulation of CNS development and functions. It was suggested these receptor/ligand signaling may function in the central nervous system as a promoter of pain transmission, since Bv8 could causes prolonged hyperalgesia in rats. Mollay, et al., supra.). Mouse Bv8 also acts as an endogenous neurotrophic factor and supports neuronal survival by protecting cells against apoptotic and excitotoxic death. Melchiorri, et al., European J. of Neuroscience 13: 1694-1702 (2001).

### SUMMARY OF THE INVENTION

In one aspect, the invention relates to methods of screening for compounds which bind to and activate (agonist) or inhibit activation (antagonist) of monkey AXOR8, human AXOR8, and human AXOR52 (receptors) by their ligands: human BV8-a (SEQ ID NO:8), mouse BV8-a (SEQ ID NO:10), frog BV8 (SEQ ID NO:12), human BV8-b (SEQ ID NO:14), human PRO1186 (SEQ ID NO:16), human PRO1186 variant (SEQ ID NO:18), and mamba intestinal toxin (herein “MIT”) (SEQ ID NO:19). Such identified compounds can be used in the treatment of: bacterial, fungal, protozoan and viral infections, particularly infections caused by HIV-1 or HIV-2; pain; cancers; diabetes, obesity; anorexia; bulimia; asthma; Parkinson's disease; acute heart failure; hypotension; hypertension; urinary retention; osteoporosis; angina pectoris; myocardial infarction; stroke; ulcers; asthma; allergies; benign prostatic hypertrophy; migraine; vomiting; psychotic and neurological disorders, including anxiety, schizophrenia, manic depression, depression, delirium, dementia, and severe mental retardation; and dyskinesias, such as Huntington's disease or Gilles dela Tourett's syndrome.

One particularly preferred embodiment of the present invention relates to a method for identifying an agonist or antagonist of the human AXOR8 polypeptide set forth in SEQ ID NO:4, said method comprising the steps of:

(a) in the presence of a labeled or unlabeled ligand selected from the group consisting of: human BV8-a (SEQID NO:8), mouse BV8-a (SEQID NO:10), frog BV8 (SEQ ID NO:12), human BV8-b (SEQ ID NO:14), human PRO1186 (SEQ ID NO:16), human PRO1186 variant (SEQ ID NO:18), and MIT (SEQ ID NO:19), contacting a cell expressing on the surface thereof the polypeptide, said polypeptide being associated with a second component capable of providing a detectable signal in response to the binding of a compound to said polypeptide, with a compound to be screened under conditions to permit binding to the polypeptide; and

(b) determining whether the compound binds to and activates or inhibits the polypeptide by measuring the level of a signal generated from the interaction of the compound with the polypeptide.

In a second particularly preferred embodiment, the present invention relates to a method for identifying an agonist or antagonist of the human AXOR8 polypeptide set forth in SEQ ID NO:4, said method comprising the steps of:

(a) determining the inhibition of binding of a ligand selected from the group consisting of: human BV8-a (SEQ ID NO:8), mouse BV8-a (SEQ ID NO:10), frog BV8 (SEQ ID NO:12), human BV8-b (SEQ ID NO:14), human PRO1186 (SEQ ID NO:16), human PRO1186 variant (SEQ ID NO:18), and MIT (SEQ ID NO:19) to cells having the polypeptide on the surface thereof, or to cell membranes containing the polypeptide, in the presence of a candidate compound under conditions to permit binding to the polypeptide; and(b) determining the amount of ligand bound to the polypeptide, such that a compound that causes the reduction of binding of a ligand is an agonist or antagonist.

In a third particularly preferred embodiment, the present invention relates to a method for identifying an agonist or antagonist of the human AXOR52 polypeptide set forth in SEQ ID NO:6, said method comprising the steps of:

(a) in the presence of a labeled or unlabeled ligand selected from the group consisting of: human BV8-a (SEQ ID NO:8), mouse BV8-a (SEQ ID NO:10), frog BV8 (SEQ ID NO:12), human BV8-b (SEQ ID NO:14), human PRO1186 (SEQ ID NO:16), human PRO1186 variant (SEQ ID NO:18), and MIT (SEQ ID NO:19), contacting a cell expressing on the surface thereof the polypeptide, said polypeptide being associated with a second component capable of providing a detectable signal in response to the binding of a compound to said polypeptide, with a compound to be screened under conditions to permit binding to the polypeptide; and

(b) determining whether the compound binds to and activates or inhibits the polypeptide by measuring the level of a signal generated from the interaction of the compound with the polypeptide.

In a fourth particularly preferred embodiment, the present invention relates to a method for identifying an agonist or antagonist of the human AXOR52 polypeptide set forth in SEQ ID NO:6, said method comprising the steps of:

(a) determining the inhibition of binding of a ligand selected from the group consisting of: human BV8-a (SEQ ID NO:8), mouse BV8-a (SEQ ID NO:10), frog BV8 (SEQ ID NO:12), human BV8-b (SEQ ID NO:14), human PRO1186 (SEQ ID NO:16), human PRO1186 variant (SEQ ID NO:18), and MIT (SEQ ID NO:19) to cells having the polypeptide on the surface thereof, or to cell membranes containing the polypeptide, in the presence of a candidate compound under conditions to permit binding to the polypeptide; and

(b) determining the amount of ligand bound to the polypeptide, such that a compound that causes the reduction of binding of a ligand is an agonist or antagonist.

In another preferred embodiment, the present invention relates to a method of activating the AXOR8 receptor (SEQ ID NO:4) in a human in need thereof, said method comprising the step of:

administering to said human a therapeutically effective amount of an AXOR8 receptor ligand in combination with a carrier, wherein said ligand is selected from the group consisting of: human BV8-a (SEQ ID NO:8), mouse BV8-a (SEQ ID NO:10), frog BV8 (SEQ ID NO:12), human BV8-b (SEQ ID NO:14), human PRO1186 (SEQ ID NO:16), human PRO1186 variant (SEQ ID NO:18), and MIT (SEQ ID NO:19).

In yet another preferred embodiment, the present invention relates to a method of activating the AXOR52 receptor (SEQ ID NO:6) in a human in need thereof, said method comprising the step of:

administering to said human a therapeutically effective amount of an AXOR52 receptor ligand in combination with a carrier, wherein said ligand is selected from the group consisting of: human BV8-a (SEQ ID NO:8), mouse BV8-a (SEQ ID NO:10), frog BV8 (SEQ ID NO:12), human BV8-b (SEQ ID NO:14), human PRO1186 (SEQ ID NO:16), human PRO1186 variant (SEQ ID NO:18), and MIT (SEQ ID NO:19).

### BRIEF DESCRIPTION OF THE DRAWINGS

FIG. 1 shows the nucleotide sequence of monkey AXOR8 receptor (SEQ ID NO:1).

FIG. 2 shows the deduced amino acid sequence of monkey AXOR8 receptor (SEQ ID NO:2).

FIG. 3 shows the nucleotide sequence of human AXOR8 receptor (SEQ ID NO:3).

FIG. 4 shows the deduced amino acid sequence of human AXOR8 receptor (SEQ ID NO:4).

FIG. 5 shows the nucleotide sequence of human AXOR52 receptor (SEQ ID NO:5).

FIG. 6 shows the deduced amino acid sequence of human AXOR52 receptor (SEQ ID NO:6).

FIG. 7 shows the nucleotide sequence of human BV8-a (SEQ ID NO:7).

FIG. 8 shows the deduced amino acid sequence of human BV8-a (SEQ ID NO:8).

FIG. 9 shows the nucleotide sequence of mouse BV8-a (SEQ ID NO:9).

FIG. 10 shows deduced amino acid sequence of mouse BV8-a (SEQ ID NO:10).

FIG. 11 shows the nucleotide sequence of frog BV8 (SEQ ID NO:11).

FIG. 12 shows deduced amino acid sequence of frog BV8 (SEQ ID NO:12).

FIG. 13 shows the nucleotide sequence of human BV8-b (SEQ ID NO:13).

FIG. 14 shows deduced amino acid sequence of human BV8-b (SEQ ID NO:14).

FIG. 15 shows the nucleotide sequence of human PRO1186 (SEQ ID NO:15).

FIG. 16 shows the deduced amino acid sequence of human PRO1186 (SEQ ID NO:16).

FIG. 17 shows the nucleotide sequence of human PRO1186 variant (SEQ ID NO:17).

FIG. 18 shows the deduced amino acid sequence of human PRO1186 variant (SEQ ID NO:18).

FIG. 19 shows the deduced amino acid sequence of MIT (SEQ ID NO:19).

FIG. 20 shows the active fraction from the pig brain fractionation that was sequenced to give the initial sequence data for the follow-up to identify the active proteins.

FIG. 21 shows that human BV8-a (SEQ ID NO:8) activates monkey AXOR8 receptor (SEQ ID NO:2).

FIG. 22 shows that mouse BV 8-a (SEQ ID NO:10) activates monkey AXOR8 receptor (SEQ ID NO:2).

FIG. 23 shows that human PRO1186 (SEQ ID NO:16) activates monkey AXOR8 receptor (SEQ ID NO:2).

FIG. 24 shows that human PRO1186 variant (SEQ ID NO:18) activates monkey AXOR8 receptor (SEQ ID NO:2).

FIGS. 25 and 26 depict the purification of MIT (SEQ ID NO:19).

FIG. 27 shows that purified MIT (SEQ ID NO:19) activates monkey AXOR8 receptor (SEQ ID NO:2).

FIG. 28 shows that frog BV8 (SEQ ID NO:12) activates monkey AXOR8 receptor (SEQ ID NO:2).

FIG. 29 shows that human BV8-b (SEQ ID NO:14) activates monkey AXOR8 receptor (SEQ ID NO:2).

FIG. 30 shows that human BV8-a (SEQ ID NO:8) activates human AXOR52 receptor (SEQ ID NO:6).

FIG. 31 shows that mouse BV8-a (SEQ ID NO:10) activates human AXOR52 receptor (SEQ ID NO6).

FIG. 32 shows that human PRO1186 (SEQ ID NO:16) activates human AXOR52 receptor (SEQ ID NO:6).

FIG. 33 shows that human PRO1186 variant (SEQ ID NO:18) activates human AXOR52 receptor (SEQ ID NO:6).

FIGS. 34 and 35 depict the purification of MIT (SEQ ID NO:19).

FIG. 36 shows that purified MIT (SEQ ID NO:19) activates human AXOR52 receptor (SEQ ID NO:6).

FIG. 37 shows that frog BV8 (SEQ ID NO:12) activates human AXOR52 receptor (SEQ ID NO:6).

FIG. 38 shows that human BV8-b (SEQ ID NO:14) activates human AXOR52 receptor (SEQ ID NO:6).

FIG. 39 shows that MIT (SEQ ID NO:19) activates human AXOR8 receptor (SEQ ID NO:4).

FIG. 40 shows that mouse BV8-a (SEQ ID NO:10), human BV8-b (SEQ ID NO:14), human PRO1186 (SEQ ID NO:16), and human PRO1186 variant (SEQ ID NO:18) activate human AXOR8 receptor (SEQ ID NO:4).

### DESCRIPTION OF THE INVENTION

Definitions

The following definitions are provided to facilitate understanding of certain terms used frequently herein.

“Monkey AXOR8” refers generally to polypeptides having the amino acid sequence set forth in SEQ ID NO:2 or an allelic variant thereof.

“Human AXOR8” refers generally to polypeptides having the amino acid sequence set forth in SEQ ID NO:4 or an allelic variant thereof.

“Human AXOR52” refers generally to polypeptides having the amino acid sequence set forth in SEQ ID NO:6 or an allelic variant thereof.

“Human BV8-a” refers generally to polypeptides having the amino acid sequence set forth in SEQ ID NO:8 or an allelic variant thereof.

“Mouse BV8-a” refers generally to polypeptides having the amino acid sequence set forth in SEQ ID NO:10 or an allelic variant thereof.

“Frog BV8” refers generally to polypeptides having the amino acid sequence set forth in SEQ ID NO:12 or an allelic variant thereof.

“Human BV8-b” refers generally to polypeptides having the amino acid sequence set forth in SEQ ID NO:14 or an allelic variant thereof.

“PRO1186” refers generally to polypeptides having the amino acid sequence set forth in SEQ ID NO:16 or an allelic variant thereof.

“PRO1186 variant” refers generally to polypeptides having the amino acid sequence set forth in SEQ ID NO:18 or an allelic variant thereof.

“Mamba intestinal toxin (MIT)” refers generally to polypeptides having the amino acid sequence set forth in SEQ ID NO:19 or an allelic variant thereof.

“Receptor Activity” or “Biological Activity of the Receptor” refers to the metabolic or physiologic function of AXOR8 and AXOR52 receptors, including similar activities or improved activities or these activities with decreased undesirable side-effects. Also included are antigenic and immunogenic activities of said AXOR8 and AXOR52 receptors.

“Monkey AXOR8 polypeptides” refers to polypeptides with amino acid sequences sufficiently similar to monkey AXOR8, preferably exhibiting at least one biological activity of the receptor.

“Human AXOR8 polypeptides” refers to polypeptides with amino acid sequences sufficiently similar to human AXOR8, preferably exhibiting at least one biological activity of the receptor.

“Human AXOR52 polypeptides” refers to polypeptides with amino acid sequences sufficiently similar to human AXOR52, preferably exhibiting at least one biological activity of the receptor.

“Monkey AXOR8 gene” refers to a polynucleotide having the nucleotide sequence set forth in SEQ ID NO:1 or allelic variants thereof and/or their complements.

“Human AXOR8 gene” refers to a polynucleotide having the nucleotide sequence set forth in SEQ ID NO:3 or allelic variants thereof and/or their complements.

“Human AXOR52 gene” refers to a polynucleotide having the nucleotide sequence set forth in SEQ ID NO:5 or allelic variants thereof and/or their complements.

“Monkey AXOR8 polynucleotides” refers to polynucleotides containing a nucleotide sequence which encodes a monkey AXOR8 polypeptide of SEQ ID NO:2, or a nucleotide sequence which has sufficient identity to a nucleotide sequence contained in SEQ ID NO:1 to hybridize under conditions useable for amplification or for use as a probe or marker.

“Human AXOR8 polynucleotides” refers to polynucleotides containing a nucleotide sequence which encodes a human AXOR8 polypeptide of SEQ ID NO:4, or a nucleotide sequence which has sufficient identity to a nucleotide sequence contained in SEQ ID NO:3 to hybridize under conditions useable for amplification or for use as a probe or marker.

“Human AXOR52 polynucleotides” refers to polynucleotides containing a nucleotide sequence which encodes a human AXOR52 polypeptide of SEQ ID NO:6, or a nucleotide sequence which has sufficient identity to a nucleotide sequence contained in SEQ ID NO:5 to hybridize under conditions useable for amplification or for use as a probe or marker.

“Antibodies” as used herein includes polyclonal and monoclonal antibodies, chimeric, single chain, and humanized antibodies, as well as Fab fragments, including the products of an Fab or other immunoglobulin expression library.

“Isolated” means altered “by the hand of man” from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide separated from the coexisting materials of its natural state is “isolated”, as the term is employed herein.

“Polynucleotide” generally refers to any polyribonucleotide or polydeoxribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as posttranslational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. See, for instance, PROTEINS—STRUCTURE AND MOLECULAR PROPERTIES, 2nd Ed., T. E. Creighton, W. H. Freeman and Company, New York, 1993 and Wold, F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York, 1983; Seifter, et al., “Analysis for protein modifications and nonprotein cofactors”, Meth Enzymol (1990) 182:626-646 and Rattan, et al., “Protein Synthesis: Posttranslational Modifications and Aging”, Ann NY Acad Sci (1992) 663:48-62.

“Variant” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis.

“Identity” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988)). Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al., Nucleic Acids Research 12(1): 387 (1984)), BLASTP, BLASTN, and FASTA (Atschul, S. F., et al., J. Molec. Biol. 215: 403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

Preferred parameters for polypeptide sequence comparison include the following:

1) Algorithm: Needleman and Wunsch, J. Mol Biol. 48: 443-453 (1970)

Comparison matrix: BLOSSUM62 from Hentikoff and Hentikoff, Proc. Natl. Acad. Sci. USA. 89:10915-10919 (1992)

Gap Penalty: 12

Gap Length Penalty: 4

A program useful with these parameters is publicly available as the “gap” program from Genetics Computer Group, Madison Wis. The aforementioned parameters are the default parameters for peptide comparisons (along with no penalty for end gaps).

Preferred parameters for polynucleotide comparison include the following:

1) Algorithm: Needleman and Wunsch, J. Mol Biol. 48: 443-453 (1970)

Comparison matrix: matches=+10, mismatch=0

Gap Penalty: 50

Gap Length Penalty: 3

Available as: The “gap” program from Genetics Computer Group, Madison Wis. These are the default parameters for nucleic acid comparisons.

By way of example, a polynucleotide sequence of the present invention may be identical to the reference sequence of SEQ ID NO:2, that is it may be 100% identical, or it may include up to a certain integer number of amino acid alterations as compared to the reference sequence such that the percent identity is less than 100% identity. Such alterations are selected from the group consisting of at least one nucleic acid deletion, substitution,, including transition and transversion, or insertion, and wherein said alterations may occur at the 5′ or 3′ terminal positions of the reference polynucleotide sequence or anywhere between those terminal positions, interspersed either individually among the nucleic acids in the reference sequence or in one or more contiguous groups within the reference sequence. The number of nucleic acid alterations for a given percent identity is determined by multiplying the total number of amino acids in SEQ ID NO:2 by the integer defining the percent identity divided by 100 and then subtracting that product from said total number of amino acids in SEQ ID NO:2, or:

nₙ=xₙ−(xₙy),

wherein nₙ is the number of amino acid alterations, xₙ is the total number of amino acids in SEQ ID NO:2, y is, for instance 0.70 for 70%, 0.80 for 80%, 0.85 for 85% etc., is the symbol for the multiplication operator, and wherein any non-integer product of xₙ and y is rounded down to the nearest integer prior to subtracting it from xₙ.

Preferred polypeptide embodiments further include an isolated polypeptide comprising a polypeptide having at least a 50,60, 70, 80, 85, 90, 95, 97 or 100% identity to a polypeptide reference sequence of SEQ ID NO:2, wherein said polypeptide sequence may be identical to the reference sequence of SEQ ID NO:2 or may include up to a certain integer number of amino acid alterations as compared to the reference sequence, wherein said alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence, and wherein said number of amino acid alterations is determined by multiplying the total number of amino acids in SEQ ID NO:2 by the integer defining the percent identity divided by 100 and then subtracting that product from said total number of amino acids in SEQ ID NO:2, or:

nₐxₐ−(xₐy),

wherein nₐ is the number of amino acid alterations, xₐ is the total number of amino acids in SEQ ID NO:2, y is 0.50 for 50%, 0.60 for 60%, 0.70 for 70%, 0.80 for 80%, 0.85 for 85%, 0.90 for 90%, 0.95 for 95%, 0.97 for 97% or 1.00 for 100%, and is the symbol for the multiplication operator, and wherein any non-integer product of xₐ and y is rounded down to the nearest integer prior to subtracting it from xₐ.

By way of example, a polypeptide sequence of the present invention may be identical to the reference sequence of SEQ ID NO:2, that is it may be 100% identical, or it may include up to a certain integer number of amino acid alterations as compared to the reference sequence such that the percent identity is less than 100% identity. Such alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in SEQ ID NO:2 by the integer defining the percent identity divided by 100 and then subtracting that product from said total number of amino acids in SEQ ID NO:2, or:

nₐ=xₐ−(xₐy),

wherein nₐ is the number of amino acid alterations, xₐ is the total number of amino acids in SEQ ID NO:2, y is, for instance 0.70 for 70%, 0.80 for 80%, 0.85 for 85%, etc., and is the symbol for the multiplication operator, and wherein any non-integer product of xₐ and y is rounded down to the nearest integer prior to subtracting it from xₐ.

Polypeptides of the Invention

The monkey and human AXOR8 polypeptides of the present invention include the polypeptides of SEQ ID NOs:2 and 4, respectively (in particular, the mature polypeptides). Such AXOR8 polypeptides may be in the form of the “mature” protein or may be a part of a larger protein such as a fusion protein. The human AXOR52 polypeptides of the present invention include the polypeptide of SEQ ID NO:6 (in particular, the mature polypeptides). Such AXOR52 polypeptides may be in the form of the “mature” protein or may be a part of a larger protein such as a fusion protein. It is often advantageous to include an additional amino acid sequence which contains secretory or leader sequences, pro-sequences, sequences which aid in purification such as multiple histidine residues, or an additional sequence for stability during recombinant production.

Biologically active fragments of the monkey AXOR8, human AXOR8, and human AXOR52 polypeptides are also included in the invention. A fragment is a polypeptide having an amino acid sequence that entirely is the same as part, but not all, of the amino acid sequence of the aforementioned polypeptides. As with monkey AXOR8, human AXOR8, and human AXOR52 polypeptides, fragments may be “free-standing,” or comprised within a larger polypeptide of which they form a part or region, most preferably as a single continuous region. Representative examples of polypeptide fragments of the invention, include, for example, fragments from about amino acid number 1-20, 21-40, 41-60, 61-80, 81-100, and 101 to the end of monkey AXOR8, human AXOR8,and human AXOR52 polypeptides. In this context “about” includes the particularly recited ranges larger or smaller by several, 5, 4, 3, 2 or 1 amino acid at either extreme or at both extremes.

Preferred fragments include, for example, truncation polypeptides having the amino acid sequence of monkey AXOR8, human AXOR8, and human AXOR52 polypeptides, except for deletion of a continuous series of residues that includes the amino terminus, or a continuous series of residues that includes the carboxyl terminus or deletion of two continuous series of residues, one including the amino terminus and one including the carboxyl terminus. Also preferred are fragments characterized by structural or functional attributes such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions. Biologically active fragments are those that mediate receptor activity, including those with a similar activity or an improved activity, or with a decreased undesirable activity. Also included are those that are antigenic or immunogenic in an animal, especially in a human.

Thus, the polypeptides of the invention include polypeptides having the amino acid sequences set forth in SEQ ID NOs:2, 4, and 6. Preferably, all of these polypeptides retain the biological activity of the receptor, including antigenic activity. Included in this group are variants of the defined sequence and fragments. Preferred variants are those that vary from the referents by conservative amino acid substitutions—i.e., those that substitute a residue with another of like characteristics. Typical such substitutions are among Ala, Val, Leu and Ile; among Ser and Thr; among the acidic residues Asp and Glu; among Asn and Gln; and among the basic residues Lys and Arg; or aromatic residues Phe and Tyr. Particularly preferred are variants in which several, 5-10, 1-5, or 1-2 amino acids are substituted, deleted, or added in any combination.

The monkey AXOR8, human AXOR8, and human AXOR52 polypeptides of the invention can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

Polynucleotides of the Invention

Another aspect of the invention relates to isolated polynucleotides which encode monkey and human AXOR8 polypeptides, as well as polynucleotides closely related thereto.

Monkey and human AXOR8 receptors are structurally related to other proteins of the G-Protein coupled receptor family. Human AXOR8 receptor (SEQ ID NO:4) shares 27.5% amino acid sequence identity and 38.7 amino acid sequence similarity with the neuropeptide Y receptor 2, a G protein-coupled receptor (Gerald, et al., J Biol Chem. 270(45): 26758-61 (1995); Rose, et al., J Biol Chem. 270(39):22661-4 (1995)). Human AXOR8 receptor (SEQ ID NO:4) shares 26.4% amino acid sequence identity and 36.5% amino acid sequence similarity with GPR10, a G protein-coupled receptor (Marchese, et al., Genomics 29(2):335-44 (1995)). Human AXOR8 receptor (SEQ ID NO:4) shares 29.2% sequence identity and 39.6% amino acid similarity with the orexin receptor, a G protein-coupled receptor (Sakurai, et al., Cell 92: 573-585 (1998)).

Monkey AXOR8 receptor (SEQ ID NO:2) shares 27.5% amino acid sequence identity and 38.7 amino acid sequence similarity with the neuropeptide Y receptor 2, a G protein-coupled receptor (Gerald, et al., supra; Rose, et al., supra). Monkey AXOR8 receptor (SEQ ID NO:2) shares 26.4% amino acid sequence identity and 36.5% amino acid sequence similarity with GPR10, a G protein-coupled receptor (Marchese, et al., supra). Monkey AXOR8 receptor (SEQ ID NO:2) shares 28.7% sequence identity and 39.1% amino acid similarity with the orexin receptor, a G protein-coupled receptor (Sakurai, et al, supra).

The nucleotide sequence of monkey AXOR8 receptor (SEQ ID NO:1) shares 97.0% sequence identity with the nucleotide sequence of human AXOR8 (SEQ ID NO:3). The amino acid sequence of monkey AXOR8 receptor (SEQ ID NO:2) shares 98.9% sequence identity with the amino acid sequence of human AXOR8 (SEQ ID NO:4).

Human AXOR52 receptor (SEQ ID NO:6) shares 27.8% amino acid sequence identity and 39.6% amino acid sequence similarity with the neuropeptide Y receptor 2, a G protein-coupled receptor (Gerald, et al., supra; Rose, et al., supra). Human AXOR52 receptor (SEQ ID NO:6) shares 28.4% amino acid sequence identity and 39.0% amino acid sequence similarity with GPR10, a G protein-coupled receptor (Marchese, et al., supra). Human AXOR52 receptor (SEQ ID NO:6) shares 29.6% sequence identity and 40.0% amino acid similarity with the orexin receptor, a G protein-coupled receptor (Sakurai, et al. supra).

Moreover, human AXOR8 receptor and human AXOR52 receptor share a high degree of homology. The nucleotide sequence of human AXOR8 receptor (SEQ ID NO:3) shares 81.7% sequence identity with the nucleotide sequence of human AXOR52 receptor (SEQ ID NO:5). The amino acid sequence of human AXOR8 receptor (SEQ ID NO:4) shares 84.1% sequence identity with the amino acid sequence of human AXOR52 receptor (SEQ ID NO:6).

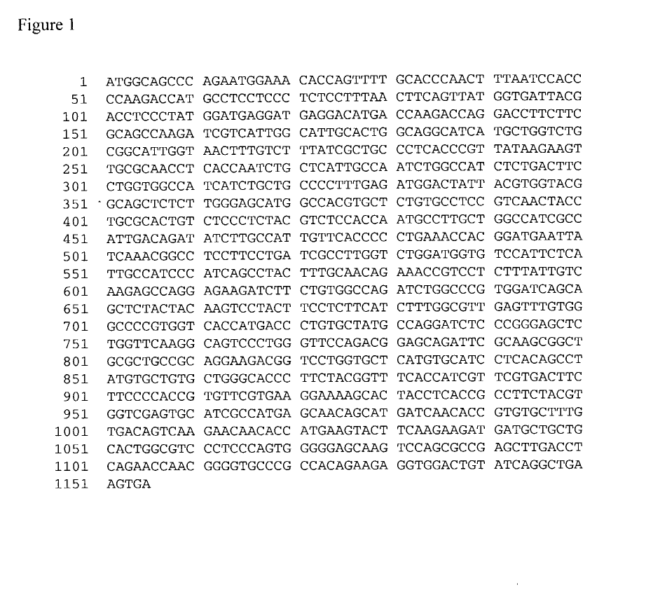

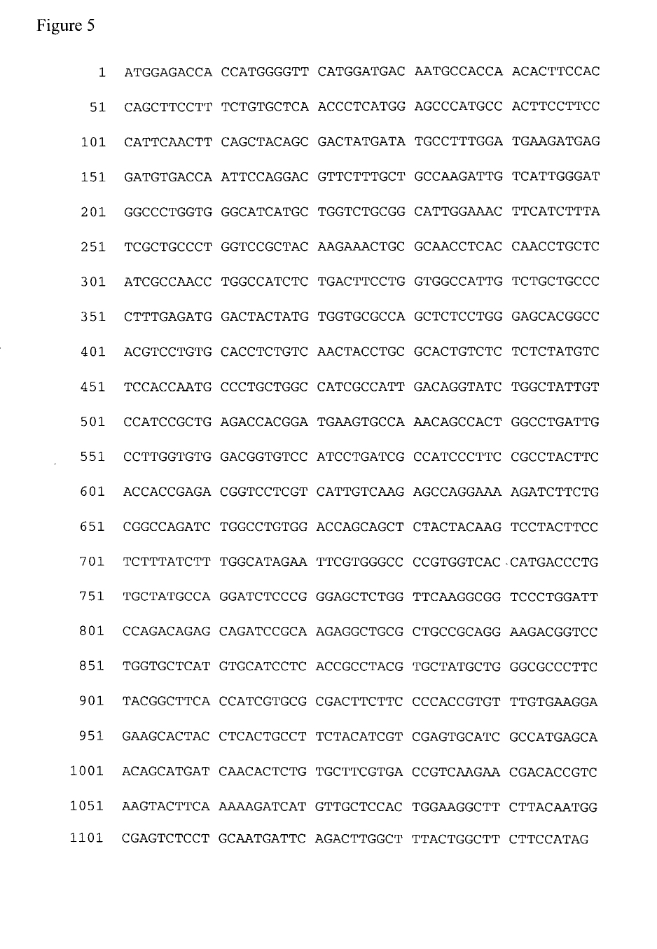

The nucleotide sequence encoding monkey and human AXOR8 polypeptides may be identical over their entire length to the coding sequence in FIGS. 1 (SEQ ID NO:1) and 3 (SEQ ID NO:3), respectively. The nucleotide sequence encoding human AXOR52 polypeptides may be identical over their entire length to the coding sequence in FIG. 5 (SEQ ID NO:5).

When the polynucleotides of the invention are used for the recombinant production of monkey AXOR8, human AXOR8, or human AXOR52 polypeptides, the polynucleotides may include the coding sequence for the mature polypeptide or a fragment thereof, by itself; the coding sequence for the mature polypeptide or fragment in reading frame with other coding sequences, such as those encoding a leader or secretory sequence, a pre-, or pro- or prepro-protein sequence, or other fusion peptide portions. For example, a marker sequence which facilitates purification of the fused polypeptide can be encoded. In certain preferred embodiments of this aspect of the invention, the marker sequence is a hexa-histidine peptide, as provided in the pQE vector (Qiagen, Inc.) and described in Gentz, et al., Proc Natl Acad Sci USA (1989) 86:821-824, or is an HA tag. The polynucleotide may also contain non-coding 5′ and 3′ sequences, such as transcribed, non-translated sequences, splicing and polyadenylation signals, ribosome binding sites and sequences that stabilize mRNA.

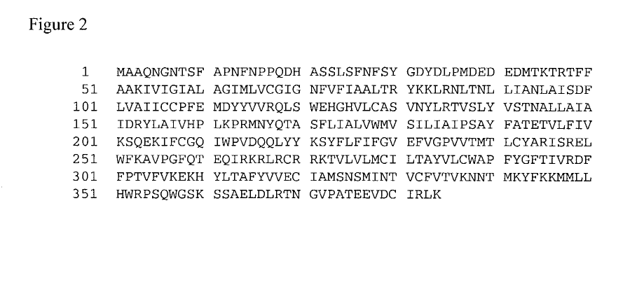

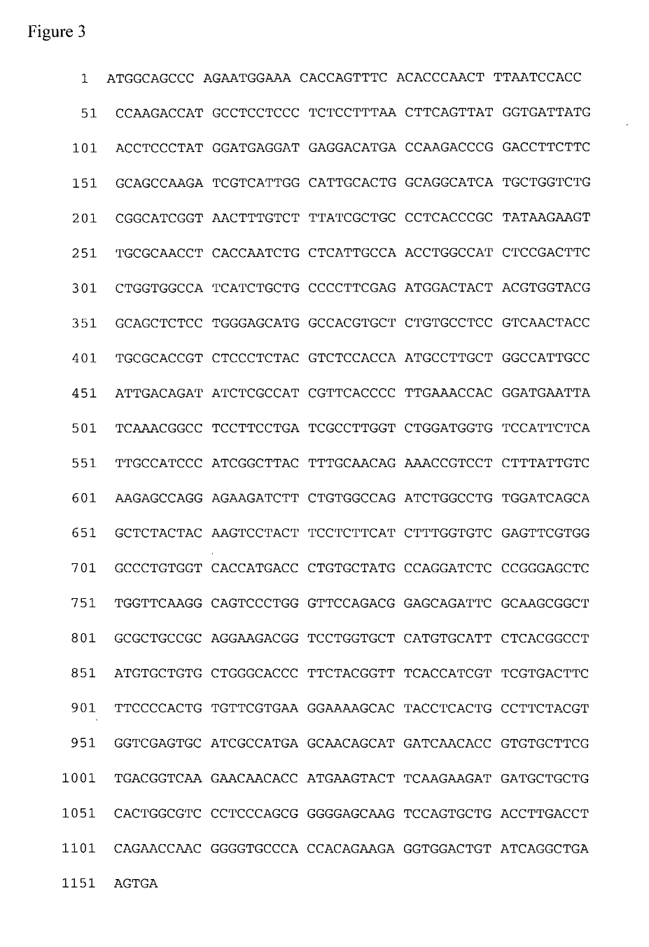

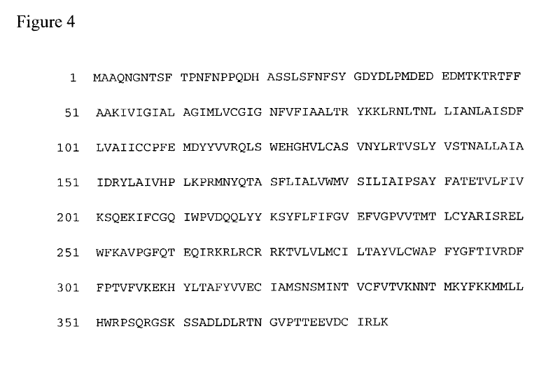

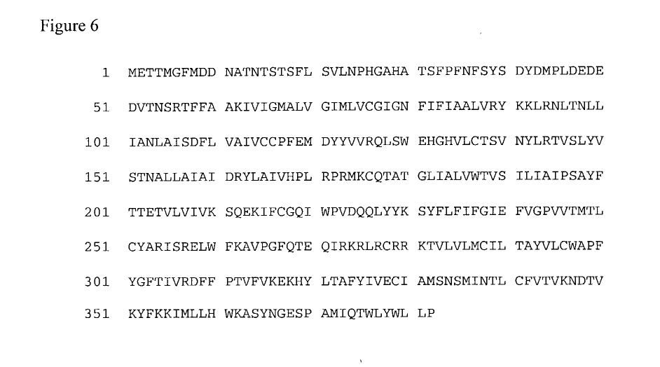

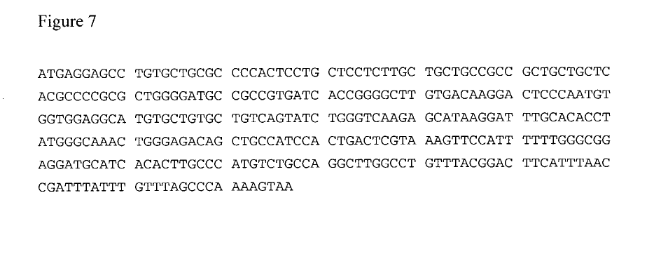

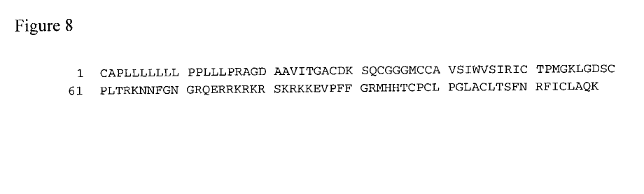

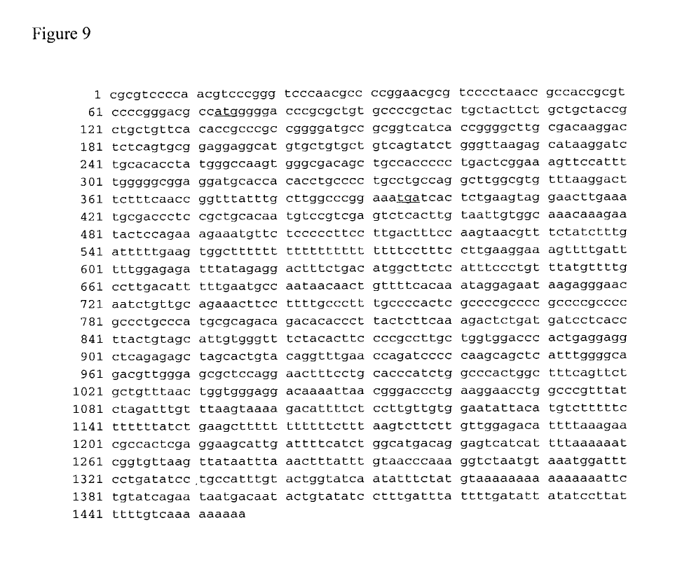

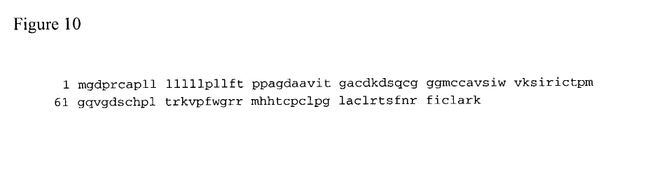

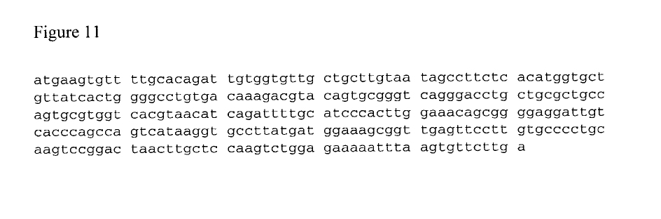

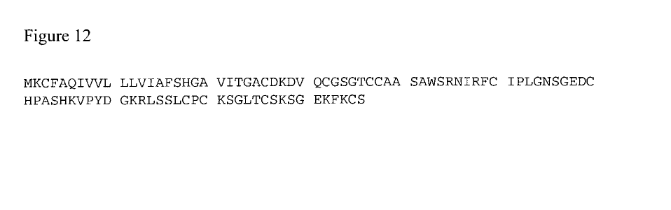

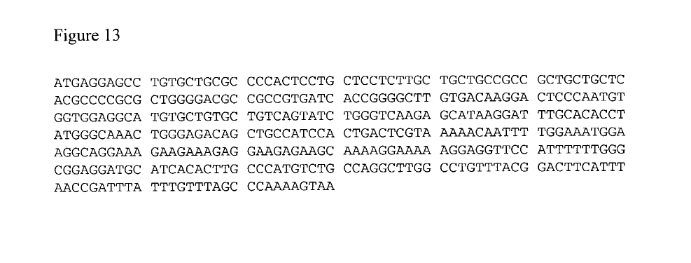

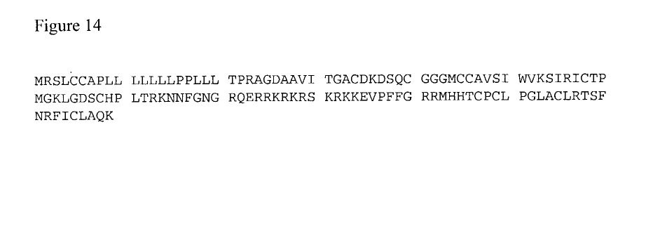

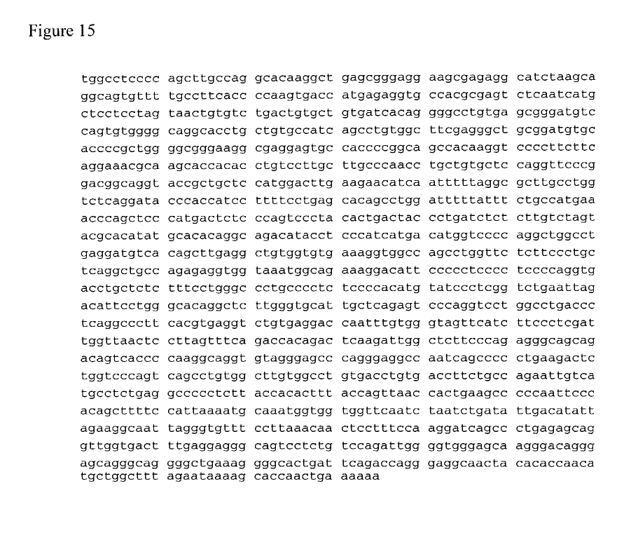

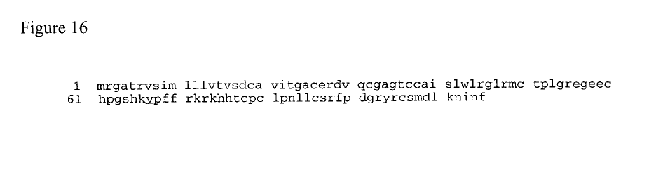

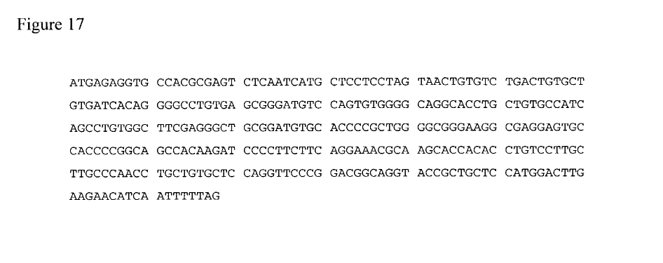

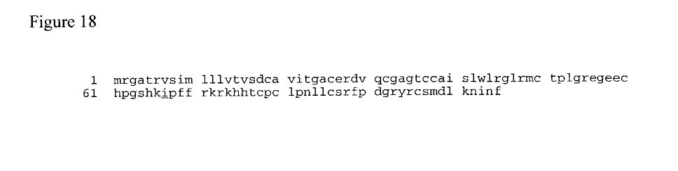

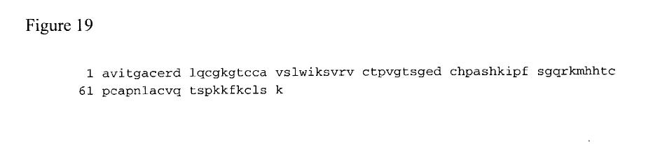

Among particularly preferred embodiments of the invention are polynucleotides encoding monkey and human AXOR8 polypeptides having the amino acid sequences set out in FIGS. 2 (SEQ ID NO:2) and 4 (SEQ ID NO:4), respectively, as well as variants thereof. Further particularly preferred embodiments of the invention are polynucleotides encoding human AXOR52 polypeptides having the amino acid sequence set out in FIG. 6 (SEQ ID NO:6), respectively, as well as variants thereof

Further preferred embodiments are polynucleotides encoding monkey or human AXOR8 variants that have the amino acid sequences of FIG. 2 (SEQ ID NO:2) or 4 (SEQ ID NO:4), respectively, in which several, 5-10, 1-5, 1-3, 1-2 or 1 amino acid residues are substituted, deleted or added, in any combination. Other preferred embodiments are polynucleotides encoding human AXOR52 variants that have the amino acid sequence of FIG. 6 (SEQ ID NO:6), respectively, in which several, 5-10, 1-5, 1-3, 1-2 or 1 amino acid residues are substituted, deleted or added, in any combination.

Vectors, Host Cells, Expression

The present invention also relates to vectors which comprise a polynucleotide or polynucleotides of the present invention, and host cells which are genetically engineered with vectors of the invention and to the production of polypeptides of the invention by recombinant techniques. Cell-free translation systems can also be employed to produce such proteins using RNAs derived from the DNA constructs of the present invention.

For recombinant production, host cells can be genetically engineered to incorporate expression systems or portions thereof for polynucleotides of the present invention. Introduction of polynucleotides into host cells can be effected by methods described in many standard laboratory manuals, such as Davis, et al., BASIC METHODS IN MOLECULAR BIOLOGY (1986) and Sambrook, et al., MOLECULAR CLONING: A LABORATOR/Y MANUAL, 2nd Ed., Cold Spring Harbor Laboratory Press, Cold Spring Harbor, N.Y. (1989) such as calcium phosphate transfection, DEAE-dextran mediated transfection, transvection, microinjection, cationic lipid-mediated transfection, electroporation, transduction, scrape loading, ballistic introduction or infection.

Representative examples of appropriate hosts include bacterial cells, such as streptococci, staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, 293 and Bowes melanoma cells; and plant cells.

A great variety of expression systems can be used. Such systems include, among others, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector suitable to maintain, propagate or express polynucleotides to produce a polypeptide in a host may be used. The appropriate nucleotide sequence may be inserted into an expression system by any of a variety of well-known and routine techniques, such as, for example, those set forth in Sambrook, et al., MOLECULAR CLONING, A LABORATORY MANUAL (supra).

For secretion of the translated protein into the lumen of the endoplasmic reticulum, into the periplasmic space or into the extracellular environment, appropriate secretion signals may be incorporated into the desired polypeptide. These signals may be endogenous to the polypeptide or they may be heterologous signals.

If the monkey AXOR8, human AXOR8, or human AXOR52 polypeptide is to be expressed for use in screening assays, generally, it is preferred that the polypeptide be produced at the surface of the cell. In this event, the cells may be harvested prior to use in the screening assay. If monkey AXOR8, human AXOR8, or human AXOR52 polypeptide is secreted into the medium, the medium can be recovered in order to recover and purify the polypeptide; if produced intracellularly, the cells must first be lysed before the polypeptide is recovered.

Monkey AXOR8, human AXOR8, or human AXOR52 polypeptides can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding proteins may be employed to regenerate active conformation when the polypeptide is denatured during isolation and or purification.

Antibodies

The polypeptides of the invention or their fragments or analogs thereof, or cells expressing them can also be used as immunogens to produce antibodies to the monkey AXOR8, human AXOR8, or human AXOR52 polypeptides.

Antibodies generated against monkey AXOR8, human AXOR8, or human AXOR52 polypeptides can be obtained by administering the polypeptides or epitope-bearing fragments, analogs or cells to an animal, preferably a nonhuman, using routine protocols. For preparation of monoclonal antibodies, any technique which provides antibodies produced by continuous cell line cultures can be used. Examples include the hybridoma technique (Kohler, G. and Milstein, C., Nature (1975) 256:495-497), the trioma technique, the human B-cell hybridoma technique (Kozbor, et al., Immunology Today (1983) 4:72) and the EBV-hybridoma technique (Cole, et al., MONOCLONAL ANTIBODIES AND CANCER THERAPY, pp. 77-96, Alan R. Liss, Inc., 1985).

Techniques for the production of single chain antibodies (U.S. Pat. No. 4,946,778) can also be adapted to produce single chain antibodies to polypeptides of this invention. Also, transgenic mice, or other organisms including other mammals, may be used to express humanized antibodies.

The above-described antibodies may be employed to isolate or to identify clones expressing the polypeptide or to purify the polypeptides by affinity chromatography.

Antibodies against human AXOR8 and human AXOR52 polypeptides may also be employed to treat bacterial, fungal, protozoan and viral infections, particularly infections caused by HIV-1 or HIV-2; pain; cancers; diabetes, obesity; anorexia; bulimia; asthma; Parkinson's disease; acute heart failure; hypotension; hypertension; urinary retention; osteoporosis; angina pectoris; myocardial infarction; stroke; ulcers; asthma; allergies; benign prostatic hypertrophy; migraine; vomiting; psychotic and neurological disorders, including anxiety, schizophrenia, manic depression, depression, delirium, dementia, and severe mental retardation; and dyskinesias, such as Huntington's disease or Gilles dela Tourett's syndrome.

Screening Assays

Monkey AXOR8, human AXOR8, or human AXOR52 polypeptides (receptor of the present invention) may be employed in a screening process for compounds which bind the receptor and which activate (agonists) or inhibit activation of (antagonists) the receptor polypeptide of the present invention. Thus, polypeptides of the invention may also be used to assess the binding of small molecule substrates and ligands in, for example, cells, cell-free preparations, chemical libraries, and natural product mixtures. These substrates and ligands may be natural substrates and ligands or may be structural or functional mimetics. See Coligan, et al., Current Protocols in Immunology 1(2):Chapter 5 (1991).

AXOR8 and AXOR52 proteins are ubiquitous in the mammalian host and are responsible for many biological functions, including many pathologies. Accordingly, it is desirous to find compounds and drugs which stimulate AXOR8 or AXOR52, on the one hand, and which can inhibit the function of AXOR8 or AXOR52, on the other hand. In general, agonists are employed for therapeutic and prophylactic purposes for such conditions as bacterial, fungal, protozoan and viral infections, particularly infections caused by HIV-1 or HIV-2; pain; cancers; diabetes, obesity; anorexia; bulimia; asthma; Parkinson's disease; acute heart failure; hypotension; hypertension; urinary retention; osteoporosis; angina pectoris; myocardial infarction; stroke; ulcers; asthma; allergies; benign prostatic hypertrophy; migraine; vomiting; psychotic and neurological disorders, including anxiety, schizophrenia, manic depression, depression, delirium, dementia, and severe mental retardation; and dyskinesias, such as Huntington's disease or Gilles dela Tourett's syndrome. Antagonists may be employed for a variety of therapeutic and prophylactic purposes for such conditions as bacterial, fungal, protozoan and viral infections, particularly infections caused by HIV-1 or HIV-2; pain; cancers; diabetes, obesity; anorexia; bulimia; asthma; Parkinson's disease; acute heart failure; hypotension; hypertension; urinary retention; osteoporosis; angina pectoris; myocardial infarction; stroke; ulcers; asthma; allergies; benign prostatic hypertrophy; migraine; vomiting; psychotic and neurological disorders, including anxiety, schizophrenia, manic depression, depression, delirium, dementia, and severe mental retardation; and dyskinesias, such as Huntington's disease or Gilles dela Tourett's syndrome.

In general, such screening procedures involve providing appropriate cells which express a receptor polypeptide of the present invention on the surface thereof. Such cells include cells from mammals, yeast, Drosophila or E. coli. In particular, a polynucleotide encoding the receptor of the present invention is employed to transfect cells to thereby express monkey AXOR8, human AXOR8, or AXOR52 polypeptide. The expressed receptor is then contacted with a test compound to observe binding, stimulation or inhibition of a functional response.

One such screening procedure involves the use of melanophores which are transfected to express the monkey AXOR8, human AXOR8, or human AXOR52 polypeptide. Such a screening technique is described in PCT WO 92/01810, published Feb. 6, 1992. Such an assay may be employed to screen for a compound which inhibits activation of a receptor of the present invention by contacting the melanophore cells which encode the receptor with both a receptor and a ligand, such as: human BV8-a (SEQ ID NO:8), mouse BV8-a (SEQ ID NO:10), frog BV8 (SEQ ID NO:12), human BV8-b (SEQ ID NO:14), human PRO1186 (SEQ ID NO:16), human PRO1186 variant (SEQ ID NO:18), or MIT (SEQ ID NO:19), and a compound to be screened. Inhibition of the signal generated by the ligand indicates that a compound is a potential antagonist for the receptor, i.e., inhibits activation of the receptor.

The technique may also be employed for screening of compounds which activate a receptor of the present invention by contacting such cells with compounds to be screened and determining whether such compound generates a signal, i.e., activates the receptor.

Other screening techniques include the use of cells which express monkey AXOR8, human AXOR8, or human AXOR52 polypeptide receptor (for example, transfected CHO cells) in a system which measures extracellular pH changes caused by receptor activation. In this technique, compounds may be contacted with cells expressing a receptor polypeptide of the present invention. A second messenger response, e.g., signal transduction or pH changes, is then measured to determine whether the potential compound activates or inhibits the receptor.

Yet another screening technique involves expressing monkey AXOR8, human AXOR8, or human AXOR52 polypeptide in which the receptor is linked to phospholipase C or D. Representative examples of such cells include, but are not limited to, endothelial cells, smooth muscle cells, and embryonic kidney cells. The screening may be accomplished as hereinabove described by detecting activation of the receptor or inhibition of activation of the receptor from the phospholipase second signal.

Another method involves screening for compounds which are antagonists, and thus inhibit activation of a receptor polypeptide of the present invention by determining inhibition of binding of labeled ligand, such as human BV8-a or mouse BV8-a to cells which have the receptor on the surface thereof, or cell membranes containing the monkey AXOR8, human AXOR8, or human AXOR52 receptor. Such a method involves transfecting a eukaryotic cell with a DNA encoding a monkeyAXOR8, human AXOR8, or human AXOR52 polypeptide such that the cell expresses the receptor on its surface. The cell is then contacted with a potential antagonist in the presence of a labeled form of a ligand, such as: human BV8-a (SEQ ID NO:8), mouse BV8-a (SEQ ID NO:10), frog BV8 (SEQ ID NO:12), human BV8-b (SEQ ID NO:14), human PRO1186 (SEQ ID NO:16), human PRO1186 variant (SEQ ID NO:18), or MIT (SEQ ID NO:19). The ligand can be labeled, e.g., by radioactivity. The amount of labeled ligand bound to the receptors is measured, e.g., by measuring radioactivity associated with transfected cells or membrane from these cells. If the compound binds to the receptor, the binding of labeled ligand to the receptor is inhibited as determined by a reduction of labeled ligand which binds to the receptors. This method is called binding assay.

Another such screening procedure involves the use of mammalian cells which are transfected to express a receptor of the present invention. The cells are loaded with an indicator dye that produces a fluorescent signal when bound to calcium, and the cells are contacted with a test substance and a receptor agonist, such as: human BV8-a (SEQ ID NO:8), mouse BV8-a (SEQ ID NO:10), frog BV8 (SEQ ID NO:12), human BV8-b (SEQ ID NO:14), human PRO1186 (SEQ ID NO:16), human PRO1186 variant (SEQ ID NO:18), or MIT (SEQ ID NO:19). Any change in fluorescent signal is measured over a defined period of time using, for example, a fluorescence spectrophotometer or a fluorescence imaging plate reader. A change in the fluorescence signal pattern generated by the ligand indicates that a compound is a potential antagonist (or agonist) for the receptor.

Another such screening procedure involves use of mammalian cells which are transfected to express a receptor of the present invention, and which are also transfected with a reporter gene construct that is coupled to activation of the receptor (for example, luciferase or beta-galactosidase behind an appropriate promoter). The cells are contacted with a test substance and a receptor agonist, such as: human BV8-a (SEQ ID NO:8), mouse BV8-a (SEQ ID NO:10), frog BV8 (SEQ ID NO:12), human BV8-b (SEQ ID NO:14), human PRO1186 (SEQ ID NO:16), human PRO1186 variant (SEQ ID NO:18), or MIT (SEQ ID NO:19), and the signal produced by the reporter gene is measured after a defined period of time. The signal can be measured using a luminometer, spectrophotometer, fluorimeter, or other such instrument appropriate for the specific reporter construct used. Inhibition of the signal generated by the ligand indicates that a compound is a potential antagonist for the receptor.

Another such screening technique for antagonists or agonists involves introducing RNA encoding monkey AXOR8, human AXOR8, or human AXOR52 polypeptide into Xenopus oocytes to transiently or stably express the receptor. The receptor oocytes are then contacted with a receptor ligand, such as: human BV8-a (SEQ ID NO:8), mouse BV8-a (SEQ ID NO:10), frog BV8 (SEQ ID NO:12), human BV8-b (SEQ ID NO:14), human PRO1186 (SEQ ID NO:16), human PRO1186 variant (SEQ ID NO:18), or MIT (SEQ ID NO:19), and a compound to be screened. Inhibition or activation of the receptor is then determined by detection of a signal, such as cAMP, calcium, proton, or other ions.

Another method involves screening for monkey AXOR8, human AXOR8, or human AXOR52 polypeptide inhibitors by determining inhibition or stimulation of monkey AXOR8, human AXOR8, or AXOR52 polypeptide-mediated cAMP and/or adenylate cyclase accumulation or dimunition. Such a method involves transiently or stably transfecting a eukaryotic cell with monkey AXOR8, human AXOR8, or human AXOR52 polypeptide to express the receptor on the cell surface. The cell is then exposed to potential antagonists in the presence of an AXOR8 or AXOR52 polypeptide ligand, such as: human BV8-a (SEQ ID NO:8), mouse BV8-a (SEQ ID NO:10), frog BV8 (SEQ ID NO:12), human BV8-b (SEQ ID NO:14), human PRO1186 (SEQ ID NO:16), human PRO1186 variant (SEQ ID NO:18), or MIT (SEQ ID NO:19). The amount of cAMP accumulation is then measured, for example, by radio-immuno or protein binding assays (e.g., using Flashplates or a scintillation proximity assay). Changes in cAMP levels can also be determined by directly measuring the activity of the enzyme, adenylyl cyclase, in broken cell preparations. If the potential antagonist binds the receptor, and thus inhibits AXOR8 or AXOR52 polypeptide binding, the levels of AXOR8 or AXOR52 polypeptide-mediated cAMP, or adenylate cyclase activity, will be reduced or increased.

Another screening method for agonists and antagonists relies on the endogenous pheromone response pathway in the yeast, Saccharomyces cerevisiae. Heterothallic strains of yeast can exist in two mitotically stable haploid mating types, MATa and MATα. Each cell type secretes a small peptide hormone that binds to a G-protein coupled receptor on opposite mating-type cells which triggers a MAP kinase cascade leading to GI arrest as a prelude to cell fusion. Genetic alteration of certain genes in the pheromone response pathway can alter the normal response to pheromone, and heterologous expression and coupling of human G-protein coupled receptors and humanized G-protein subunits in yeast cells devoid of endogenous pheromone receptors can be linked to downstream signaling pathways and reporter genes (e.g., U.S. Pat. Nos. 5,063,154; 5,482,835; 5,691,188). Such genetic alterations include, but are not limited to, (i) deletion of the STE2 or STE3 gene encoding the endogenous G-protein coupled pheromone receptors; (ii) deletion of the FAR1 gene encoding a protein that normally associates with cyclin-dependent kinases leading to cell cycle arrest; and (iii) construction of reporter genes fused to the FUS1 gene promoter (where FUS1 encodes a membrane-anchored glycoprotein required for cell fusion). Downstream reporter genes can permit either a positive growth selection (e.g., histidine prototrophy using the FUS1-HIS3 reporter), or a calorimetric, fluorimetric or spectrophotometric readout, depending on the specific reporter construct used (e.g., b-galactosidase induction using a FUS1-LacZ reporter).

The yeast cells can be further engineered to express and secrete small peptides from random peptide libraries, some of which can permit autocrine activation of heterologously expressed human (or mammalian) G-protein coupled receptors (Broach, J. R. and Thorner, J. Nature 384: 14-16, 1996; Manfredi, et al., Mol. Cell. Biol. 16: 4700-4709, 1996). This provides a rapid direct growth selection (e.g,, using the FUS1-HIS3 reporter) for surrogate peptide agonists that activate characterized or orphan receptors. Alternatively, yeast cells that functionally express human (or mammalian) G-protein coupled receptors linked to a reporter gene readout (e.g., FUS1-LacZ) can be used as a platform for high-throughput screening of known ligands, fractions of biological extracts and libraries of chemical compounds for either natural or surrogate ligands. Functional agonists of sufficient potency (whether natural or surrogate) can be used as screening tools in yeast cell-based assays for identifying G-protein coupled receptor antagonists. For this purpose, the yeast system offers advantages over mammalian expression systems due to its ease of utility and null receptor background (lack of endogenous G-protein coupled receptors) which often interferes with the ability to identify agonists or antagonists.

The present invention also provides a method for determining whether a ligand not known to be capable of binding to a monkey AXOR8, human AXOR8, or human AXOR52 polypeptide can bind to such receptor which comprises contacting a yeast or mammalian cell that expresses a monkey AXOR8, human AXOR8, or human AXOR52 polypeptide with a ligand, such as: human BV8-a (SEQ ID NO:8), mouse BV8-a (SEQ ID NO:10), frog BV8 (SEQ ID NO:12), human BV8-b (SEQ ID NO:14), human PRO1186 (SEQ ID NO:16), human PRO1186 variant (SEQ ID NO:18), or MIT (SEQ ID NO:19), under conditions permitting binding of candidate ligands to a monkey AXOR8, human AXOR8, or human AXOR52 polypeptide, and detecting the presence of a candidate ligand which binds to the receptor, thereby determining whether the ligand binds to the AXOR8 or AXOR52 polypeptide. The systems hereinabove described for determining agonists and/or antagonists may also be employed for determining ligands which bind to the receptor.

The present invention also contemplates agonists and antagonists obtainable from the above described screening methods.

Examples of potential monkey AXOR8, human AXOR8, or human AXOR52 polypeptide antagonists include antibodies or, in some cases, oligonucleotides, which bind to the receptor but do not elicit a second messenger response such that the activity of the receptor is prevented. Potential antagonists also include compounds which are closely related to a ligand of the monkey AXOR8, human AXOR8, or human AXOR52 polypeptide, i.e. a fragment of the ligand, which has lost biological function and when binding to the monkey AXOR8, human AXOR8, or human AXOR52 polypeptide, elicits no response.

Thus, in another aspect, the present invention relates to a screening kit for identifying agonists, antagonists, and ligands for monkey or human AXOR8 polypeptides, which comprises:

(a) a monkey or human AXOR8 polypeptide, preferably that of SEQ ID NO:2 or SEQ ID NO:4; and further preferably comprises a labeled or unlabeled ligand selected from the group consisting of: human BV8-a (SEQ ID NO:8), mouse BV8-a (SEQ ID NO:10), frog BV8 (SEQ ID NO:12), human BV8-b (SEQ ID NO:14), human PRO1186 (SEQ ID NO:16), human PRO1186 variant (SEQ ID NO:18), and MIT (SEQ ID NO:19);

(b) a recombinant cell expressing a monkey or human AXOR8 polypeptide, preferably that of SEQ ID NO:2 or SEQ ID NO:4; and further preferably comprises a labeled or unlabeled ligand selected from the group consisting of: human BV8-a (SEQ ID NO:8), mouse BV8-a (SEQ ID NO:10), frog BV8 (SEQ ID NO:12), human BV8-b (SEQ ID NO:14), human PRO1186 (SEQ ID NO:16), human PRO1186 variant (SEQ ID NO:18), and MIT (SEQ ID NO:19); or

(c) a cell membrane expressing monkey or human AXOR8 polypeptide; preferably that of SEQ ID NO:2 or SEQ ID NO:4; and further preferably comprises a labeled or unlabeled ligand selected from the group consisting of: human BV8-a (SEQ ID NO:8), mouse BV8-a (SEQ ID NO:10), frog BV8 (SEQ ID NO:12), human BV8-b (SEQ ID NO:14), human PRO1186 (SEQ ID NO:16), human PRO1186 variant (SEQ ID NO:18), and MIT (SEQ ID NO:19).

It will be appreciated that in any such kit, (a), (b), or (c) may comprise a substantial component.

In yet another aspect, the present invention relates to a screening kit for identifying agonists, antagonists, and ligands for human AXOR52 polypeptides, which comprises:

(a) human AXOR52 polypeptide, preferably that of SEQ ID NO:6; and further preferably comprises a labeled or unlabeled ligand selected from the group consisting of: human BV8-a (SEQID NO:8), mouse BV8-a (SEQ ID NO:10), frog BV8 (SEQ ID NO:12), human BV8-b (SEQ ID NO:14), human PRO1186 (SEQ ID NO:16), human PRO1186 variant (SEQ ID NO:18), and MIT (SEQ ID NO:19);.

(b) a recombinant cell expressing human AXOR52 polypeptide, preferably that of SEQ ID NO:6; and further preferably comprises a labeled or unlabeled ligand selected from the group consisting of: human BV8-a (SEQ ID NO:8), mouse BV8-a (SEQ ID NO:10), frog BV8 (SEQ ID NO:12), human BV8-b (SEQ ID NO:14), human PRO1186 (SEQ ID NO:16), human PRO1186 variant (SEQ ID NO:18), and MIT (SEQ ID NO:19); or

(c) a cell membrane expressing human AXOR52 polypeptide; preferably that of SEQ ID NO:6; and further preferably comprises a labeled or unlabeled ligand selected from the group consisting of: human BV8-a (SEQ ID NO:8), mouse BV8-a (SEQ ID NO:10), frog BV8 (SEQ ID NO:12), human BV8-b (SEQ ID NO:14), human PRO1186 (SEQ ID NO:16), human PRO1186 variant (SEQ ID NO:18), and MIT (SEQ ID NO:19).

It will be appreciated that in any such kit, (a), (b), or (c) may comprise a substantial component.

Potential antagonists also include soluble forms of monkey AXOR8, human AXOR8, or human AXOR52 polypeptide receptor, e.g., fragments of the receptor, which bind to the ligand and prevent the ligand from interacting with membrane bound monkey AXOR8, human AXOR8, or human AXOR52 polypeptide receptors.

A potential antagonist also includes an antisense construct prepared through the use of antisense technology. Antisense technology can be used to control gene expression through triple-helix formation or antisense DNA or RNA, both methods of which are based on binding of a polynucleotide to DNA or RNA. For example, the 5′ coding portion of the polynucleotide sequence, which encodes for the mature polypeptides of the present invention, is used to design an antisense RNA oligonucleotide of from about 10 to 40 base pairs in length. A DNA oligonucleotide is designed to be complementary to a region of the gene involved in transcription (triple helix—see Lee, et al. Nucl. Acids Res., 6: 3073 (1979); Cooney, et al, Science, 241: 456 (1988); and Dervan, et al., Science, 251: 1360 (1991)), thereby preventing transcription and production of an AXOR8 receptor polypeptide. The antisense RNA oligonucleotide hybridizes to the mRNA in vivo and blocks translation of the mRNA molecule to AXOR8 polypeptide (antisense—Okano, J., Neurochem., 56: 560 (1991); OLIGODEOXYNUCLEOTIDES AS ANTISENSE INHIBITORS OF GENE EXPRESSION, CRC Press, Boca Raton, Fla. (1988)). The oligonucleotides described above can also be delivered to cells such that the antisense RNA or DNA may be expressed in vivo to inhibit production of a monkey AXOR8, human AXOR8, or human AXOR52 polypeptide.

Prophylactic and Therapeutic Methods

This invention provides methods of treating an abnormal conditions related to both an excess of and insufficient amounts of human AXOR8 or human AXOR52 receptor activity.

If the activity of human AXOR8 or human AXOR52 receptor is in excess, several approaches are available. One approach comprises administering to a subject an inhibitor compound (antagonist) as hereinabove described along with a pharmaceutically acceptable carrier in an amount effective to inhibit activation by blocking binding of ligands to the human AXOR8 or human AXOR52 receptor, or by inhibiting a second signal, and thereby alleviating the abnormal condition.

In another approach, soluble forms of human AXOR8 or human AXOR52 polypeptides still capable of binding the ligand in competition with endogenous human AXOR8 may be administered. Typical embodiments of such competitors comprise fragments of the human AXOR8 polypeptide.

In still another approach, expression of the gene encoding endogenous human AXOR8 or human AXOR52 receptor can be inhibited using expression blocking techniques. Known such techniques involve the use of antisense sequences, either internally generated or separately administered. See, for example, O'Connor, J Neurochem (1991) 56:560 in Oligodeoxynucleotides as Antisense Inhibitors of Gene Expression, CRC Press, Boca Raton, Fla. (1988). Alternatively, oligonucleotides which form triple helices with the gene can be supplied. See, for example, Lee, et al., Nucleic Acids Res (1979) 6:3073; Cooney et al., Science (1988) 241:456; Dervan, et al., Science (1991) 251:1360. These oligomers can be administered per se or the relevant oligomers can be expressed in vivo.

For treating abnormal conditions related to an under-expression of human AXOR8 or human AXOR52 receptor and its activity, several approaches are also available. One approach comprises administering to a subject a therapeutically effective amount of a compound which activates human AXOR8 or human AXOR52 receptor, i.e., an agonist as described above, in combination with a pharmaceutically acceptable carrier, to thereby alleviate the abnormal condition. Alternatively, gene therapy may be employed to effect the endogenous production of human AXOR8 or human AXOR52 receptor by the relevant cells in the subject. For example, a polynucleotide of the invention may be engineered for expression in a replication defective retroviral vector, as discussed above. The retroviral expression construct may then be isolated and introduced into a packaging cell transduced with a retroviral plasmid vector containing RNA encoding a polypeptide of the present invention such that the packaging cell now produces infectious viral particles containing the gene of interest. These producer cells may be administered to a subject for engineering cells in vivo and expression of the polypeptide in vivo. For overview of gene therapy, see Chapter 20, Gene Therapy and other Molecular Genetic-based Therapeutic Approaches, (and references cited therein) in Human Molecular Genetics, T Strachan and A P Read, BIOS Scientific Publishers Ltd. (1996).

In another embodiment, the instant invention provides a method of activating the AXOR8 or AXOR52 receptor in a human by administering to said human a therapeutically effective amount of an AXOR8 or AXOR52 receptor ligand in combination with a carrier, wherein said ligand is selected from the group consisting of: human BV8-a (SEQ ID NO:8), mouse BV8-a (SEQ ID NO:10), frog BV8 (SEQ ID NO:12), human BV8-b (SEQ ID NO:14), human PRO1186 (SEQ ID NO:16), human PRO1186 variant (SEQ ID NO:18), and MIT (SEQ ID NO:19). Administering a human AXOR8 or AXOR52 receptor ligand in this manner can be used for the treatment of human diseases/disorders, including, but not limited to: infections such as bacterial, fungal, protozoan and viral infections, particularly infections such as bacterial, fungal, protozoan and viral infections, particularly infections caused by HIV-1 or HIV-2; pain; cancers; diabetes, obesity; anorexia; bulimia; asthma; Parkinson's disease; acute heart failure; hypotension; hypertension; urinary retention; osteoporosis; angina pectoris; myocardial infarction; stroke; ulcers; asthma; allergies; benign prostatic hypertrophy; migraine; vomiting; psychotic and neurological disorders, including anxiety, schizophrenia, manic depression, depression, delirium, dementia, and severe mental retardation; and dyskinesias, such as Huntington's disease or Gilles dela Tourett's syndrome.

Formulation and Administration

Peptides, such as the soluble form of human AXOR8 or human AXOR52 polypeptides, and agonists and antagonist peptides or small molecules, may be formulated in combination with a suitable pharmaceutical carrier. Such formulations comprise a therapeutically effective amount of the polypeptide or compound, and a pharmaceutically acceptable carrier or excipient. Such carriers include but are not limited to, saline, buffered saline, dextrose, water, glycerol, ethanol, and combinations thereof. Formulation should suit the mode of administration, and is well within the skill of the art. The invention further relates to pharmaceutical packs and kits comprising one or more containers filled with one or more of the ingredients of the aforementioned compositions of the invention.

Polypeptides and other compounds of the present invention may be employed alone or in conjunction with other compounds, such as therapeutic compounds.

Preferred forms of systemic administration of the pharmaceutical compositions include injection, typically by intravenous injection. Other injection routes, such as subcutaneous, intramuscular, or intraperitoneal, can be used. Alternative means for systemic administration include transmucosal and transdermal administration using penetrants such as bile salts or fusidic acids or other detergents. In addition, if properly formulated in enteric or encapsulated formulations, oral administration may also be possible. Administration of these compounds may also be topical and/or localized, in the form of salves, pastes, gels and the like.

The dosage range required depends on the choice of peptide, the route of administration, the nature of the formulation, the nature of the subject's condition, and the judgment of the attending practitioner. Suitable dosages, however, are in the range of 0.1-100 μg/kg of subject. Wide variations in the needed dosage, however, are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization, as is well understood in the art.

Polypeptides used in treatment can also be generated endogenously in the subject, in treatment modalities often referred to as “gene therapy” as described above. Thus, for example, cells from a subject may be engineered with a polynucleotide, such as a DNA or RNA, to encode a polypeptide ex vivo, and for example, by the use of a retroviral plasmid vector. The cells are then introduced into the subject.

### EXAMPLES

### Example 1

Ligand Bank for Binding and Functional Assays

A bank of over 200 putative receptor ligands has been assembled for screening. The bank comprises: transmitters, hormones and chemokines known to act via a human seven transmembrane (7TM) receptor; naturally occurring compounds which may be putative agonists for a human 7TM receptor, non-mammalian, biologically active peptides for which a mammalian counterpart has not yet been identified; and compounds not found in nature, but which activate 7TM receptors with unknown natural ligands. This bank is used to initially screen the receptor for known ligands, using both functional (i.e., calcium, cAMP, microphysiometer, oocyte electrophysiology, etc., see below) as well as binding assays.

### Example 2

Ligand Binding Assays

Ligand binding assays provide a direct method for ascertaining receptor pharmacology and are adaptable to a high throughput format. The purified ligand for a receptor is radiolabeled to high specific activity (50-2000 Ci/mmol) for binding studies. A determination is then made that the process of radiolabeling does not diminish the activity of the ligand towards its receptor. Assay conditions for buffers, ions, pH and other modulators such as nucleotides are optimized to establish a workable signal to noise ratio for both membrane and whole cell receptor sources. For these assays, specific receptor binding is defined as total associated radioactivity minus the radioactivity measured in the presence of an excess of unlabeled competing ligand. Where possible, more than one competing ligand is used to define residual nonspecific binding.

### Example 3

Functional Assay in Xenopus Oocytes

Capped RNA transcripts from linearized plasmid templates encoding the receptor cDNAs of the invention are synthesized in vitro with RNA polymerases in accordance with standard procedures. In vitro transcripts are suspended in water at a final concentration of 0.2 mg/ml. Ovarian lobes are removed from adult female toads, Stage V defolliculated oocytes are obtained, and RNA transcripts (10 ng/oocyte) are injected in a 50 nl bolus using a microinjection apparatus. Two electrode voltage clamps are used to measure the currents from individual Xenopus oocytes in response to agonist exposure. Recordings are made in Ca2+ free Barth's medium at room temperature. The Xenopus system can be used to screen known ligands and tissue/cell extracts for activating ligands.

### Example 4

Microphysiometric Assays

Activation of a wide variety of secondary messenger systems results in extrusion of small amounts of acid from a cell. The acid formed is largely as a result of the increased metabolic activity required to fuel the intracellular signaling process. The pH changes in the media surrounding the cell are very small but are detectable by the CYTOSENSOR microphysiometer (Molecular Devices Ltd., Menlo Park, Calif.). The CYTOSENSOR is thus capable of detecting the activation of a receptor which is coupled to an energy utilizing intracellular signaling pathway such as the G-protein coupled receptor of the present invention.

### Example 5

Extract/Cell Supernatant Screening

A large number of mammalian receptors exist for which there remains, as yet, no cognate activating ligand (agonist). Thus, active ligands for these receptors may not be included within the ligands banks as identified to date. Accordingly, the 7TM receptor of the invention is also functionally screened (using calcium, cAMP, microphysiometer, oocyte electrophysiology, etc., functional screens) against tissue extracts to identify natural ligands. Extracts that produce positive functional responses can be sequentially subfractionated until an activating ligand is isolated or identified.

### Example 6

Purification of Activity to AXOR8 (Monkey and Human):

Four KG of whole porcine brain (Pelfreeze, Rogers, Ak.) stored at −70 degrees) was homogenized in 10 L of 100 mM sodium phosphate pH 2.5 containing 0.5 mM EDTA using a Waring Blender with a 4 L chamber. The tissue was processed in 500 KG portions. The resultant homogenate was further disrupted using a Tekmar Tissumizer and preparative probe for 3 minutes at a setting of 70. The final homogenate was centrifuged at 28,000×g for 30 min. at 4 degrees. The supernatant was recovered and then filtered through a N35R plastic mesh (CellMicroSieves 35 micron, BioDesign Inc., Carmel, N.Y.) and then made 0.85 M in sodium chloride. An equal volume of ethlylacetate was added to the filtrate and then it was stirred for 10 min. at room temperature. The resultant emulsion was clarified by centrifugation in a JA-10 rotor at 45000×g for 15 min. at 4 degrees. The aqueous layer was recovered and the ethylacetate layer plus any material in the interface was discarded. The aqueous layer was again filtered through a N35R mesh and then vacuum evaporated in a Rotovap instrument in order to remove any residual ethylacetate. The final evaporated sample was then filtered through a Pall SpiralCap PF Capsule (0.8/0.2 micron, Gellmann Labs, Ann Arbor, Mich.).

At this stage, the sample was divided into four equal aliquots. Each aliquot was concentrated and de-salted by chromatography on a C8 reverse-phase HPLC column (Vydac 208TP101550, 5×25 cm). The column was equilibrated in 0.06% trifluoroacetic acid in water containing 0.5 mM EDTA (HPLC buffer) and acetonitrile was used as the mobile phase in the same buffer. The column was developed using 1000 ml of 20% acetonitrile in HPLC buffer followed by a gradient of acetonitrile from 20 to 36%. The column was then washed with 100 ml of HPLC buffer containing 36% acetonitrile, further developed with a 100 ml acetonitrile gradient from 36 to 80% and then finally stripped with 750 ml of HPLC buffer containing 80% acetonitrile. Detection by absorbance at 280 nm was used to determine the material to be pooled at each step. The elution from 20 to 36% acetonitrile contained the activity and was subsequently evaporated to 100 ml using the Rotovap as before. The evaporated sample was filtered through a Sterivex 0.22 micron filter (Millipore, SVGV010RS).

Each of the four samples generated from the original homogenate were brought to pH 7 with sodium hydroxide and then fractionated on Sephadex G50 medium (5×100 cm bed). The column was equilibrated and developed at 10 ml/min. in 50 mM sodium phosphate pH 7.0, 150 mM sodium chloride and 5 mM EDTA. Aliquots of 30 ml fractions were desalted using an ASPEC XL robot (Gilson Instruments) and 6 ml C18 Sep-Pak Vac cartridges (500 mg, Waters Assoc., Milford, Mass.). The activity in the fractions was then determined using a calcium mobilization assay and the active fractions were pooled (1025 to 1475 ml elution from 2000 ml bed).

After filtration through Pall SpiralCap Capsules as before, the sample from chromatography on Sephadex G50 was further separated using ion-exchange chromatography (Source 15S, Pharmacia Biotech, 31 ml column equilibrated, loaded and washed at 8 ml/min. in 10 mM sodium phosphate pH 7.0 with 5 mM EDTA). The column was then developed first with 1 column volume of a linear increase from 0-300 mM sodium chloride in equilibration buffer, followed by a 6.5 volume gradient from 300 to 1000 mM of sodium chloride. Aliquots of fractions from ion-exchange were diluted and assayed using the calcium mobilization assay.

Reverse-Phase Chromatography: The active fractions from large-scale ion exchange chromatography were pooled and the pH was adjusted to 4. The material was then loaded onto a Jupiter C18 column (Phenomenex, 10 micron particle with 300A pore, 2.2×25 cm, 8 ml/min. flow rate) equilibrated in 5 mM sodium phosphate, 10 mM sodium citrate pH 3.5 with 1 mM EDTA. The proteins were eluted in an increasing gradient of acetonitrile in equilibration buffer and detection was accomplished by following absorption at 280 nm. Eight ml fractions were collected and then the acetonitrile was removed by evaporation in a SpeedVac concentrator (Savant Inc., Hicksville, N.Y.) for 1 hour. Active fractions were determined using the calcium mobilization assay. These were then loaded onto a diphenyl reverse-phase column using the same buffer system as for the C18 step (Vydac 219TP510, 3 ml/min. flowrate) and the active fractions were again determined by calcium mobilization assay. These were directly loaded onto an 800 microliter source 15S column equilibrated in 10 mM sodium phosphate, pH 3.2, with 10% acetonitrile and 0.2 mM EDTA. The column was developed in an increasing gradient of sodium chloride at 1 ml/min. The active fractions were determined as before and then chromatographed on a narrow-bore C18 column (Vydac 218TP52, 0.21×25 cm, 0.2 ml/min. flowrate) using a gradient of acetonitrile in 0.1% trifluoracetic acid in water.

Stability of Active Material to Monkey AXOR8 Receptor:

Initially, the active material extracted from either porcine stomach or brain was too labile to support fractionation through several steps. Therefore, conditions that would slow the loss of activity were determined. The activity used for these studies was from the first analytical C18 fractionation with the following differences. Homogenization of porcine stomach was carried out without the use of EDTA. Size exclusion was performed in 50 mM sodium phosphate, pH 2.5, and ion exchange was executed in 10 mM sodium phosphate, pH 2.7, containing 25% acetonitrile and the column was developed in the same buffer containing 1 M sodium chloride. Reverse-phase HPLC on a 2.2 cm C18 column was carried out as before but using 0.1% trifluoroacetic acid in water and acetonitrile as the mobile phase. The active fraction from reverse-phase chromatography was dried and then resuspended in 20 mM sodium phosphate at either pH 2.5 or 7.0. Agents to be evaluated for the stabilization of the activity were added at the same time. Samples were then held at 4 degrees for a predetermined number of days. Samples from day one were held for about 1 hour before they were desalted on Waters SepPak C18 cartridges. Desalting was carried out by first acidifying samples (when necessary) and then passing them over SepPaks that had been wetted in methanol and then equilibrated in 0.1% trifluoracetic acid in water. The cartridge was washed with 5 ml of the same solution and then the peptides were eluted with 60% acetonitrile in the 0.1% trifluoracetic acid. The eluted fractions were taken to dryness in a SpeedVac vacuum concentrator and subsequently resuspended in buffer for assay on FLIPR.

The results of the stability assessment indicated that EDTA was a good agent for slowing the loss of activity in crude preparations to the AXOR8 monkey receptor.

Sequencing:

Amino terminal sequencing using gas-phase Edman chemistry on the final purified activity gave the result: AVITGAcERDVQcGPGTccAVSLWLRgLRL (SEQ ID NO:20).

### Example 7

Purification of MIT-1 (SEQ ID NO:19) from Black Mamba Venom:

One hundred mg of lyophilized black mamba (Dendroaspis polyepsis) venom (Sigma, V8000) was resuspended in 50 mM sodium phosphate, pH 7.0, 150 mM sodium chloride, 5 mM EDTA, to a concentration of 5 mg/ml. The sample was fractionated on a Sephadex G-50 column (medium, 5×100 cm) at 5 ml/min and 40 ml fractions were collected. Aliquots were diluted ×5 with KRH buffer and then assayed for calcium mobilization using a FLIPR device and HEK293 cells expressing the test receptors. Peak activity fractions from chromatography on Sephadex G50 were pooled and 8% of this material was resolved over a C18 reverse phase column (Vydac 218TP510, 1×25 cm) by developing in 10 mM sodium citrate, 5 mM sodium phosphate, pH 3.5, 1 mM EDTA, at 3 ml/minute with a discontinuous gradient of 0-60% acetonitrile. 3 ml fractions were collected and lyophilized for 1 hour to remove acetonitrile. Aliquots were diluted in KRH buffer and assayed for calcium mobilization activity against the test receptors. Peak fractions of activity were pooled and 11% of this material was resolved over a Source 15S (0.5×9 cm) cation-exchange column, run at 1 ml/min. in 10 mM sodium phosphate, pH 3.25, 0.2 mM EDTA, 10% acetonitrile with a linear gradient of 0-2M sodium chloride. One-ml fractions were collected and lyophilized for 1 hour to remove acetonitrile. Aliquots were diluted as above and assayed for calcium mobilization activity as before. Fractions containing activity were examined by electrospray-MS (MicroMass LCT) and found to contain a single specie with a mass of 8507, corresponding to residues 1-80 of MIT-1 (SEQ ID NO:19).

### Example 8

Cloning and Sequencing of cDNAs that Activate the AXOR8 and AXOR52 Receptors

An activity was purified from a porcine brain extract which specifically activated Ca2+ mobilization in HEK-293 cells expressing monkey AXOR8 receptor (SEQ ID NO:2). Active fractions of this extract have been shown to specifically activate human AXOR8 receptor (SEQ ID NO:4) and human AXOR52 receptor (SEQ ID NO:6).

Peptide sequencing was performed on these porcine brain fractions. N-terminal peptide sequencing was performed on the active fraction, and 30 residues were identified. This peptide sequence was used to search the nucleotide and peptide data bases for related sequences. The sequence of the 30 amino terminal residues was similar to 2 proteins with known function: (1) BV8-a, a protein originally isolated from skin secretions of frogs (Bombina verigata) (Mollay, et al., Eur J Pharmacol. 18;374(2):189-96 (1999)); and mamba intestinal toxin (MIT), also known as Protein A (Schweitz, et al., FEBS Lett. 461(3):183-8 (1999)), a protein isolated from snake venom.

BV8 and MIT are structurally similar, sharing 44 amino acids and 9 conserved cysteine residues. These proteins are biologically active. Both proteins stimulate contraction of ileal tissue, and in rats can induce hypersensitvity to pain, or hyperalgesia, following injection into the lateral ventricle (Mollay, et al., supra). Recently, cDNAs encoding mammalian orthologues of BV8 have been identified (Jilek, et al, Gene Oct 3;256(1-2):189-95 (2000); Wecselberger, et al., FEBS Lett. 462 (1-2), 177-181 (1999); Li, et al., Mol Pharm 59(4):692-698 (2001)). Similar to the results obtained with frog BV8 and snake MIT, two human proteins similar to BV8 have been demonstrated to stimulate contraction of gastrointestinal smooth muscle. Functional and binding studies demonstrate that the actions of the mammalian orthologues of BV8 are likely mediated through a GPCR.

Using PCR, cDNAs encoding 4 proteins related to frog BV8 were cloned: (1) mouse BV8-a, (2) human PRO1186 (also referred to as prokineticin 1 by Li, et al., supra), and (3) a variant of PRO1186 which has exhibits a V to I mutation at position 67, and human BV8-a (also referred to as prokineticin 2 by Li, et al., supra). Supernatant culture fluid collected from HEK-293 cells transiently expressing these cDNAs specifically stimulated Ca2+ mobilization in HEK-293 cells transiently expressing AXOR8 or AXOR52. Mouse BV8a, PRO1186, PRO1186 variant, and human BV8-a (as well as MIT) are agonists for monkey AXOR8 receptor and human AXOR52 receptor. Therefore, these identified ligands can be used to configure high throughput screens to identify agonists and antagonists of these receptors.

Sequences Used for PCR:

Murine BV8-a and murine BV8-b were amplified with primers GSK 662 and 663 from Clontech's mouse testis Marathon library.

| | |
|---|---|
| GSK 662(forward)- | |
| 5′ ttcgccaccatgggggacccgcgct 3′ | (SEQ ID NO:21) |
| GSK 663 (reverse)- | |
| 5′ tttcatttccgggccaagcaaataaaccg 3′ | (SEQ ID NO:22) |

PRO 1186 and PRO 1186 variant were amplified from Clontech's human brain Marathon libraries with primers GSK 808 and 809:

| | |
|---|---|
| GSK 808(forward)- | |
| 5′ TTGAATTCGCCACCATGAGAGGTGCCACGCGAG | SEQ ID NO:23) |
| TC 3′ | |
| GSK 809(reverse)- | |
| 5′ TTGATATCCTAAAAATTGATGTTCTTCAAGTCC | (SEQ ID NO:24) |
| A | |

Human BV8-a was amplified from Clontech's human testis Marathon library with primers GSK 971 and 973

| | |
|---|---|
| hBv8-GSK 971(f) | |
| 5′ ttgaattcgccaccatgaggagcctgtgctgcg | (SEQ ID NO:25) |
| cc 3′ | |
| hBv8-GSK 973(r) | |
| 5′ ttgatatcttacttttgggctaaacaaataaat | (SEQ ID NO:26) |
| c 3′ | |

### Example 9

Activation of Monkey AXOR8 Receptor by its Natural Ligands

The cDNAs encoding 5 proteins related to frog BV8 were cloned using PCR: (1) mouse BV-8a (SEQ ID NO:9), (2) human PRO1186 (SEQ ID NO:15), (3) human PRO1186 variant, which has exhibits a V to I mutation at position 67 (SEQ ID NO:17), (4) human BV8-a (SEQ ID NO:7), and (5) human BV8-b (SEQ ID NO:14). In addition the frog BV8 (SEQ ID NO:11) was cloned by PCR. Supernatant culture fluid collected from HEK-293 cells transiently expressing these cDNAs individually; specifically stimulated Ca²⁺ mobilization in HEK-293 cells transiently expressing monkey AXOR8 receptor (SEQ ID NO2). Orthologs isolated from black mamba snake venom utilized the FLIPR calcium mobilization to follow the purification.

HEK 293 stable pool of monkey AXOR8 cells are loaded with Fluo-3 and tissue extract fractions and supernatants of HEK 293 cell culture fluid were evaluated for agonist induced calcium mobilization. In order to assess selectivity of the responses the same fractions were tested against HEK 293 stable pool of pCDN vector cells.

All of the supernatants expressing the above proteins induced a robust calcium response in the HEK 293 stable pool of monkey AXOR8 cells with no significant response obtained with the HEK 293 stable pool of pCDN vector cells. The purification of the protein ortholog from black mamba snake venom was followed by using the 96-well Fluorescent Imaging Plate Reader (FLIPR).

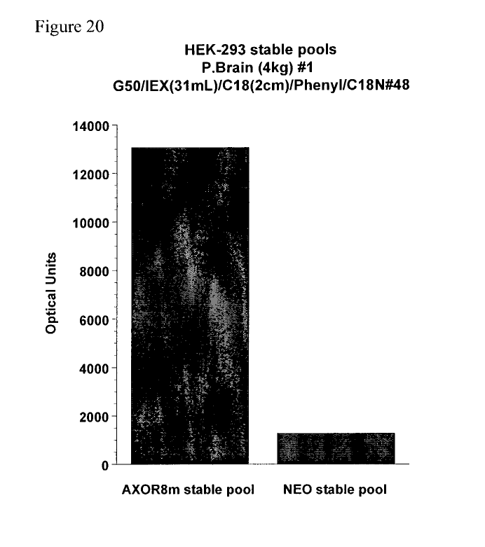

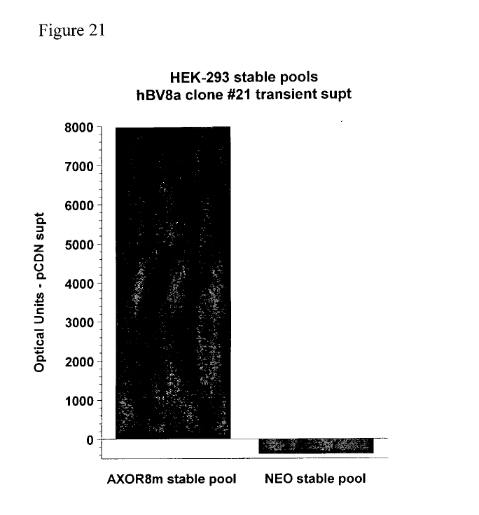

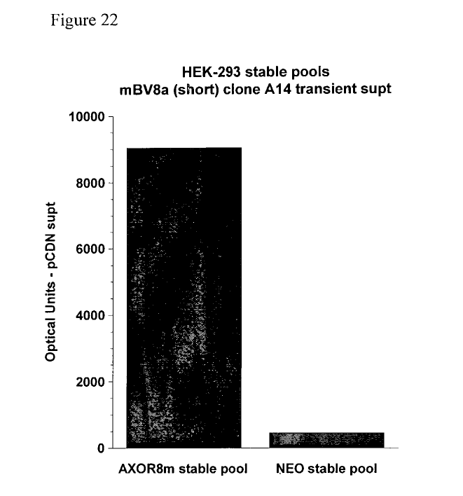

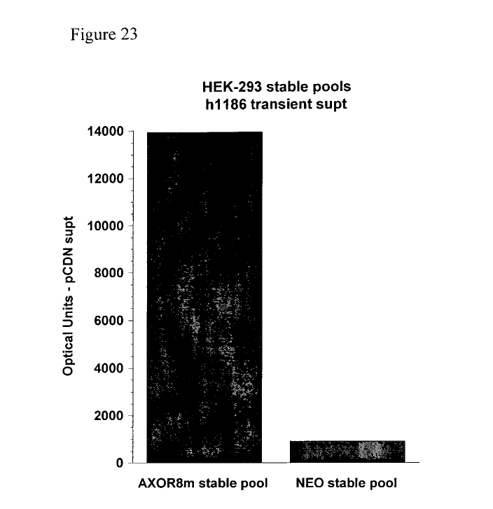

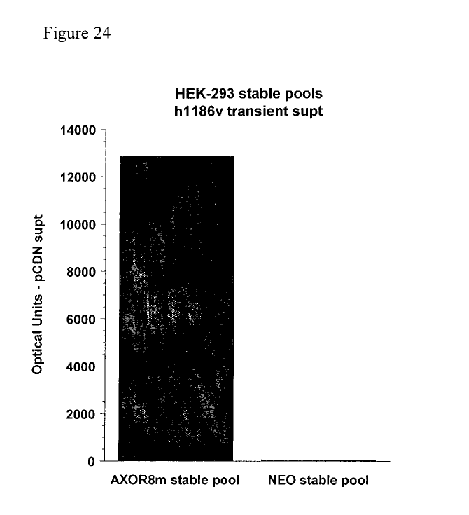

FIGS. 20-29 depict the results from these FLIPR assays. Specifically, FIG. 20 shows the active fraction from the pig brain fractionation that was sequenced to give the initial sequence data for the follow-up to identify the active proteins. FIG. 21 shows that supernatant fluid from HEK-293 cells expressing human BV8-a (SEQ ID NO:8) activates HEK-293 cells expressing monkey AXOR8 receptor (SEQ ID NO:2). FIG. 22 shows that supernatant fluid from HEK-293 cells expressing mouse BV8-a (SEQ ID NO:10) activates HEK-293 cells expressing monkey AXOR8 receptor (SEQ ID NO:2). FIG. 23 shows that supernatant fluid from HEK-293 cells expressing human PRO1186 (SEQ ID NO:16) activates HEK-293 cells expressing monkey AXOR8 receptor (SEQ ID NO:2). FIG. 24 shows that supernatant fluid from HEK-293 cells expressing human PRO1186 variant (SEQ ID NO:18) activates HEK-293 cells expressing monkey AXOR8 receptor (SEQ ID NO:2).

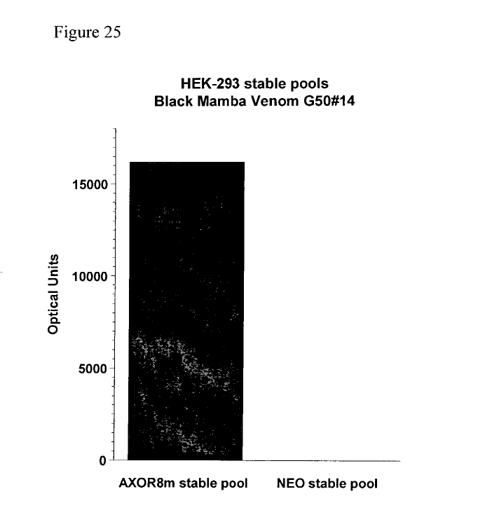

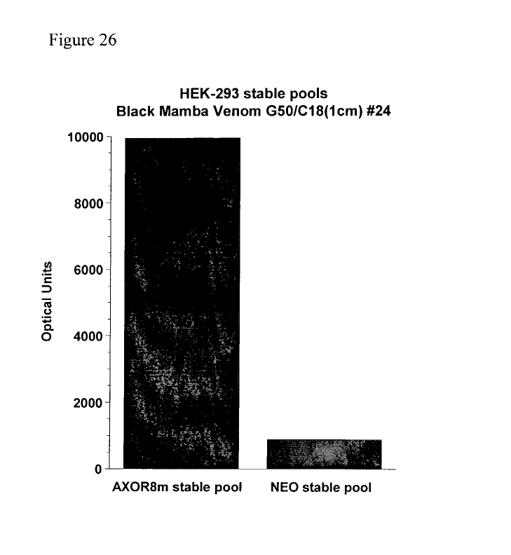

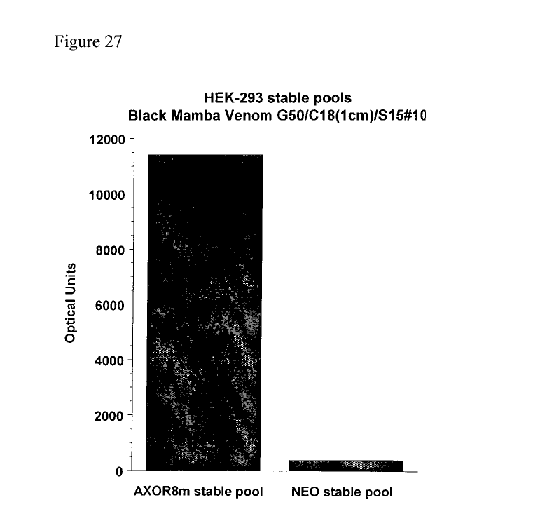

FIGS. 25-27 follow the purification of MIT (SEQ ID NO:19), the protein from the black mamba snake venom. FIG. 25 is the active fraction off the G-50 column. FIG. 26 is the active fraction off the following C18 column. FIG. 27 is the purified material off the S15 column, which shows that purified MIT (SEQ ID NO:19) activates monkey AXOR8 receptor (SEQ ID NO:2).

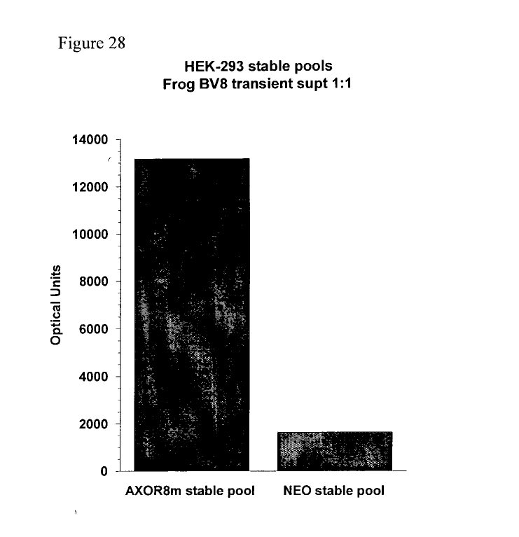

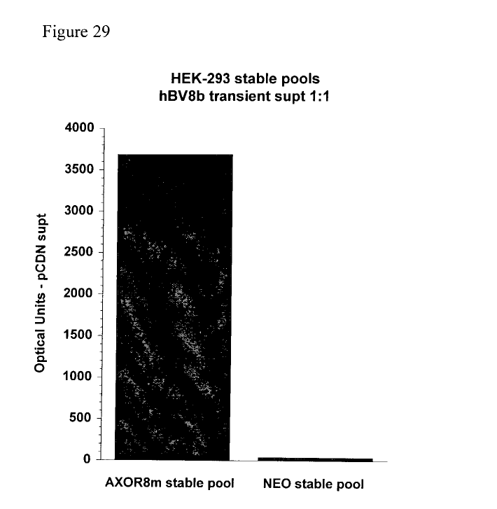

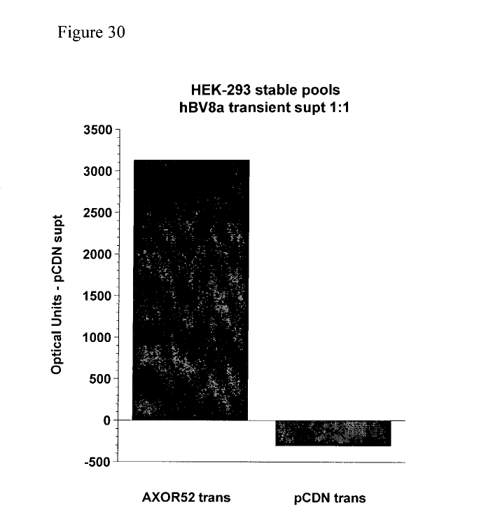

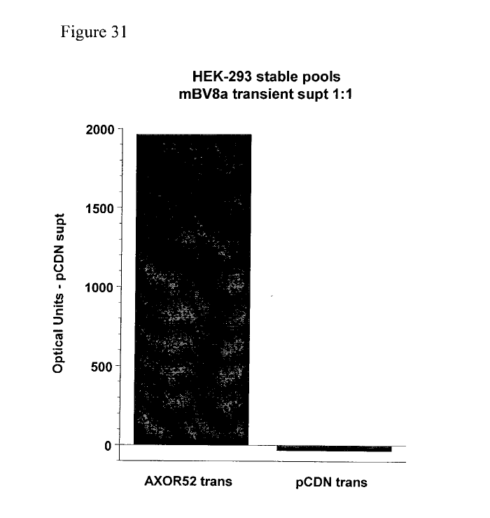

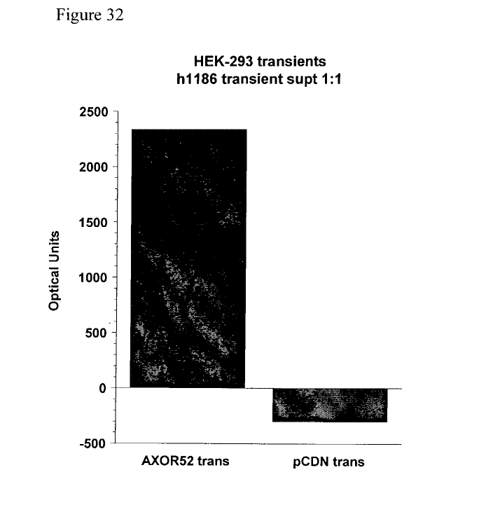

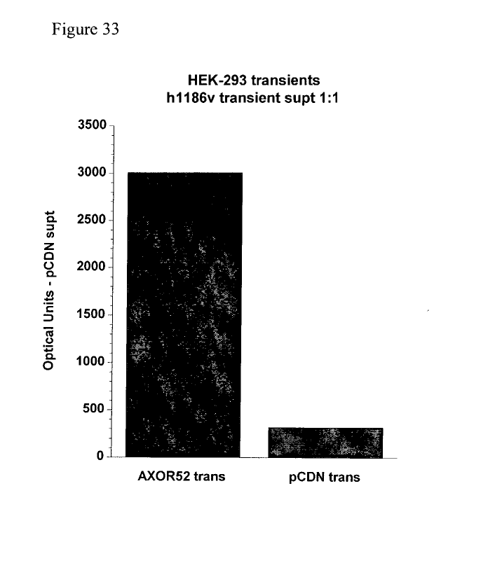

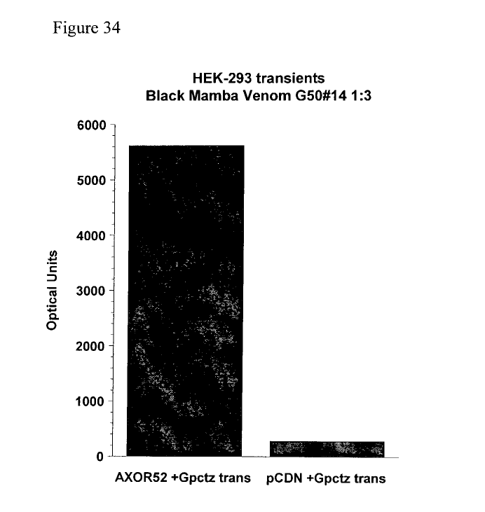

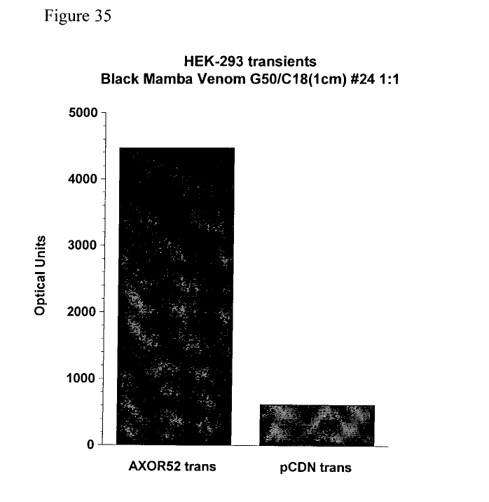

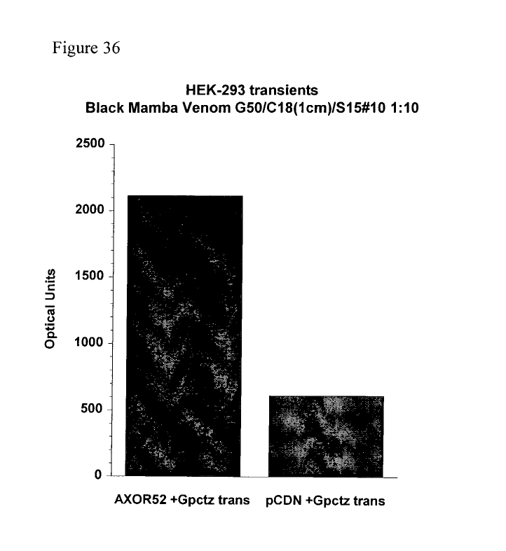

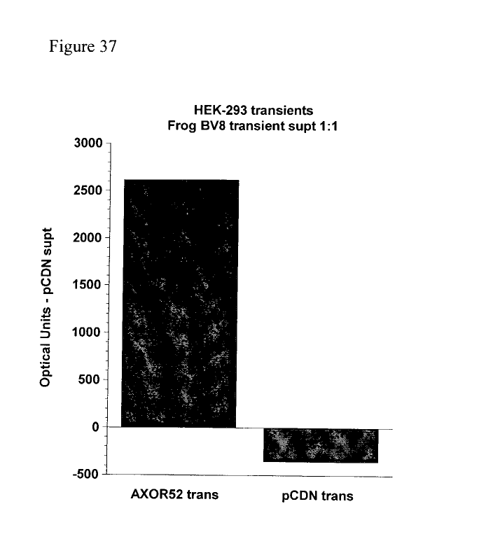

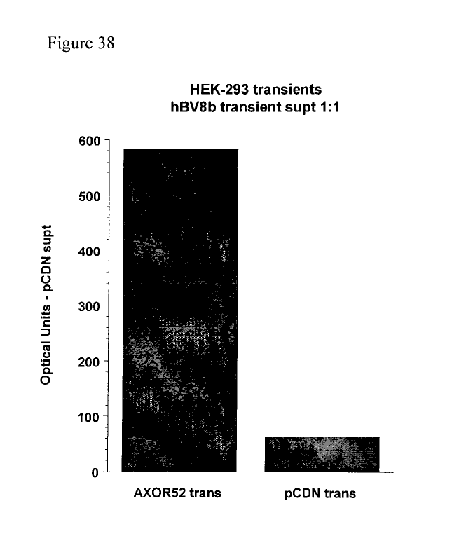

FIG. 28 shows that supernatant fluid from HEK-293 cells expressing frog BV8 (12) activates HEK-293 cells expressing monkey AXOR8 receptor (SEQ ID NO:2). FIG. 29 shows that supernatant fluid from HEK-293 cells expressing human BV8-b (SEQ ID NO:14) activates HEK-293 cells expressing monkey AXOR8 receptor (SEQ ID NO:2).

Taken together, these FLIPR results confirm that monkey AXOR8 receptor (SEQ ID NO:2) is activated by human BV8-a (SEQ ID NO:8), mouse BV8-a (SEQ ID NO:10), human PRO1186 (SEQ ID NO:16), human PRO1186 variant (SEQ ID NO:18), and MIT (SEQ ID NO:19).

### Example 11

Activation of Human AXOR8 Receptor by its Natural Ligands

The cDNAs encoding 3 proteins related to frog BV8 were cloned using PCR: (1) mouse BV8-a (SEQ ID NO:10), (2) human PRO1186 (SEQ ID NO:16) and (3) human PRO1186 variant (SEQ ID NO:18). Supernatant culture fluid collected from HEK-293 cells transiently expressing these cDNAs specifically stimulated Ca²⁺ mobilization in HEK-293 cells transiently expressing human AXOR8 receptor (SEQ ID NO:4). Orthologs isolated from black mamba snake venom utilized the FLIPR calcium mobilization to follow the purification.

HEK 293 cells transiently expressing human AXOR8 cells are loaded with Fluo-4 and tissue extract fractions and supernatants of HEK 293 cell culture fluid were evaluated for agonist induced calcium mobilization. In order to assess selectivity of the responses, the same fractions were tested against HEK 293 cells transiently expressing several other 7TM receptors.

All of the supernatants expressing the above proteins induced a robust calcium response in HEK 293 cells transiently expressing human AXOR8 with no significant response obtained in HEK 293 cells transiently expressing several other 7TM receptors. Purification of the protein ortholog from black mamba snake venom, MIT (SEQ ID NO:19) was followed using the 96-well FLIPR.

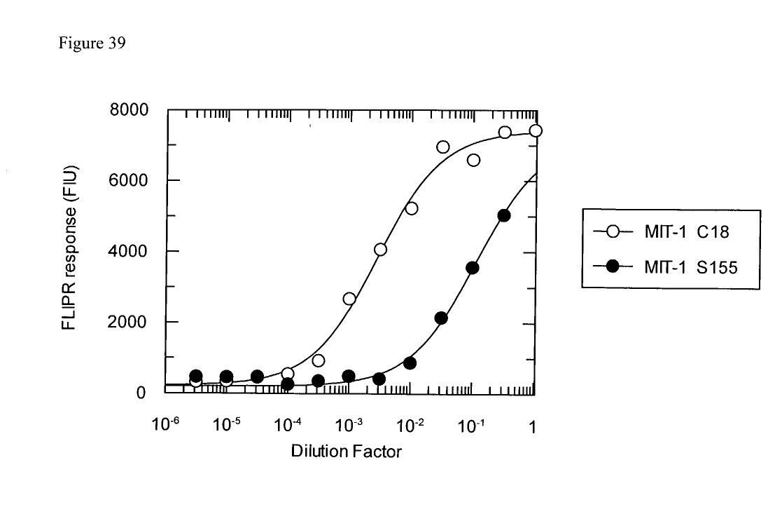

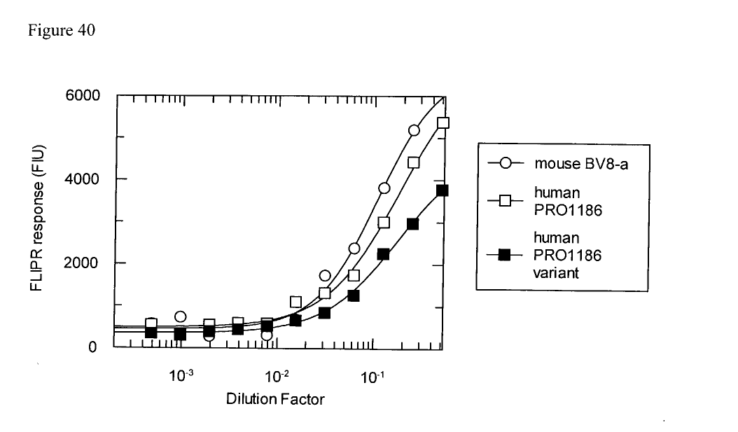

FIGS. 39 and 40 depict the results from these FLIPR assays. Specifically, FIG. 39 shows that purified fractions of MIT (SEQ ID NO:19) activate HEK-293 cells expressing human AXOR8 receptor (SEQ ID NO:4). FIG. 40 shows that supernatant fluid from HEK-293 cells expressing mouse BV8-a (SEQ ID NO:10), human PRO1186 (SEQ ID NO:16), and human PRO1186 variant (SEQ ID NO:18) activates HEK-293 cells expressing human AXOR8 receptor (SEQ ID NO:4)

### Example 12

Tissue Expression Profile (Human Taqman Data):

Human AXOR8 (SEQ ID NO:4) is expressed at low levels in brain, pituitary, heart, and bone marrow. While the highest expression of human AXOR52 (SEQ ID NO:6) is in pituitary, it is also found in brain and adipose. In brain tissue, expression of both receptors is widely distributed, with the highest level found in hypothalamus and locus coeruleus.

Human PRO1186 (SEQ ID NO:16) is expressed at the highest levels in placenta, pituitary and prostate. Human BV8-a (SEQ ID NO:8) is expressed at the highest levels in spleen, lymphocytes, and bone marrow. Human BV8-a is expressed at lower levels in lung and placenta. Both human PRO1186 and human BV8-a are also expressed in brain, with the highest expression found in hippocampus, medulla oblongata, parahippocampal gyrus.

All publications including, but not limited to, patents and patent applications, cited in this specification, are herein incorporated by reference as if each individual publication were specifically and individually indicated to be incorporated by reference herein as though fully set forth. The above description fully discloses the invention, including preferred embodiments thereof Modifications and improvements of the embodiments specifically disclosed herein are within the scope of the following claims. Without further elaboration, it is believed that one skilled in the art can, using the preceding description, utilize the present invention to its fullest extent. Therefore, the examples provided herein are to be construed as merely illustrative and are not a limitation of the scope of the present invention in any way. The embodiments of the invention in which an exclusive property or privilege is claimed are defined as follows.

What is claimed is:

1. A method for identifying an agonist or antagonist of the human AXOR8 polypeptide set forth in SEQ ID NO:4, said method comprising the steps of:
(a) in the presence of a labeled or unlabeled ligand selected from the group consisting of: human BV8-a (SEQ ID NO:8), mouse BV8-a (SEQ ID NO:10), frog BV8 (SEQ ID NO:12), human BV8-b (SEQ ID NO:14), human PRO1186 (SEQ ID NO:16), human PRO1186 variant (SEQ ID NO:18), and MIT (SEQ ID NO:19), contacting a cell expressing on the surface thereof the polypeptide, said polypeptide being associated with a second component capable of providing a detectable signal in response to the binding of a compound to said polypeptide, with a compound to be screened under conditions to permit binding to the polypeptide; and
(b) determining whether the compound binds to and activates or inhibits the polypeptide by measuring the level of a signal generated from the interaction of the compound with the polypeptide.

2. A method for identifying an agonist or antagonist of the human AXOR8 polypeptide set forth in SEQ ID NO:4, said method comprising the steps of:
(a) determining the inhibition of binding of a ligand selected from the group consisting of: human BV8-a (SEQ ID NO:8), mouse BV8-a (SEQ ID NO:10), frog BV8 (SEQ ID NO:12), human BV8-b (SEQ ID NO:14), human PRO1186 (SEQ ID NO:16), human PRO1186 variant (SEQ ID NO:18), and MIT (SEQ ID NO:19) to cells having the polypeptide on the surface thereof, or to cell membranes containing the polypeptide, in the presence of a candidate compound under conditions to permit binding to the polypeptide; and(b) determining the amount of ligand bound to the polypeptide, such that a compound that causes the reduction of binding of a ligand is an agonist or antagonist.

3. A method for identifying an agonist or antagonist of the human AXOR52 polypeptide set forth in SEQ ID NO:6, said method comprising the steps of:
(a) in the presence of a labeled or unlabeled ligand selected from the group consisting of: human BV8-a (SEQ ID NO:8), mouse BV8-a (SEQ ID NO:10), frog BV8 (SEQ ID NO:12), human BV8-b (SEQ ID NO:14), human PRO1186 (SEQ ID NO:16), human PRO1186 variant (SEQ ID NO:18), and MIT (SEQ ID NO:19), contacting a cell expressing on the surface thereof the polypeptide, said polypeptide being associated with a second component capable of providing a detectable signal in response to the binding of a compound to said polypeptide, with a compound to be screened under conditions to permit binding to the polypeptide; and
(b) determining whether the compound binds to and activates or inhibits the polypeptide by measuring the level of a signal generated from the interaction of the compound with the polypeptide.

4. A method for identifying an agonist or antagonist of the human AXOR52 polypeptide set forth in SEQ ID NO:6, said method comprising the steps of:
(a) determining the inhibition of binding of a ligand selected from the group consisting of: human BV8-a (SEQ ID NO:8), mouse BV8-a (SEQ ID NO:10), frog BV8 (SEQ ID NO:12), human BV8-b (SEQ ID NO:14), human PRO1186 (SEQ ID NO:16), human PRO1186 variant (SEQ ID NO:18), and MIT (SEQ ID NO:19) to cells having the polypeptide on the surface thereof, or to cell membranes containing the polypeptide, in the presence of a candidate compound under conditions to permit binding to the polypeptide; and
(b) determining the amount of ligand bound to the polypeptide, such that a compound that causes the reduction of binding of a ligand is an agonist or antagonist.

5. A method of activating the AXOR8 receptor (SEQ ID NO:4) in a human in need thereof, said method comprising the step of:
administering to said human a therapeutically effective amount of an AXOR8 receptor ligand in combination with a carrier, wherein said ligand is selected from the group consisting of: human BV8-a (SEQ ID NO:8), mouse BV8-a (SEQ ID NO:10), frog BV8 (SEQ ID NO:12), human BV8-b (SEQ ID NO:14), human PRO1186 (SEQ ID NO:16), human PRO1186 variant (SEQ ID NO:18), and MIT (SEQ ID NO:19).

6. A method of activating the AXOR52 receptor (SEQ ID NO:6) in a human in need thereof, said method comprising the step of:
administering to said human a therapeutically effective amount of an AXOR8 receptor ligand in combination with a carrier, wherein said ligand is selected from the group consisting of: human BV8-a (SEQ ID NO:8), mouse BV8-a (SEQ ID NO:10), frog BV8 (SEQ ID NO:12), human BV8-b (SEQ ID NO:14), human PRO1186 (SEQ ID NO:16), human PRO1186 variant (SEQ ID NO:18), and MIT (SEQ ID NO:19).


SEQUENCE LISTING (19 sequences)

SEQ ID NO: 1 - DNA, 1158 bp, Cercopithecus aethiops
guratggcagcccagaatggaaacaccagttttgcacccaactttaatccaccccaagaccatgcctcctccctctcctttaacttcagttatggtgattacgacctccctatggatgaggatgaggacatgaccaagaccaggaccttcttcgcagccaagatcgtcattggcattgcactggcaggcatcatgctggtctgcggcattggtaactttgtctttatcgctgccctcacccgttataagaagttgcgcaacctcaccaatctgctcattgccaatctggccatctctgacttcctggtggccatcatctgctgcccctttgagatggactattacgtggtacggcagctctcttgggagcatggccacgtgctctgtgcctccgtcaactacctgcgcactgtctccctctacgtctccaccaatgccttgctggccatcgccattgacagatatcttgccattgttcaccccctgaaaccacggatgaattatcaaacggcctccttcctgatcgccttggtctggatggtgtccattctcattgccatcccatcagcctactttgcaacagaaaccgtcctctttattgtcaagagccaggagaagatcttctgtggccagatctggcccgtggatcagcagctctactacaagtcctacttcctcttcatctttggcgttgagtttgtgggccccgtggtcaccatgaccctgtgctatgccaggatctcccgggagctctggttcaaggcagtccctgggttccagacggagcagattcgcaagcggctgcgctgccgcaggaagacggtcctggtgctcatgtgcatcctcacagcctatgtgctgtgctgggcacccttctacggtttcaccatcgttcgtgacttcttccccaccgtgttcgtgaaggaaaagcactacctcaccgccttctacgtggtcgagtgcatcgccatgagcaacagcatgatcaacaccgtgtgctttgtgacagtcaagaacaacaccatgaagtacttcaagaagatgatgctgctgcactggcgtccctcccagtgggggagcaagtccagcgccgagcttgacctcagaaccaacggggtgcccgccacagaagaggtggactgtatcaggctgaagtga

SEQ ID NO: 2 - protein, 384 aa, Cercopithecus aethiops
MAAQNGNTSFAPNFNPPQDHASSLSFNFSYGDYDLPMDEDEDMTKTRTFFAAKIVIGIALAGIMLVCGIGNFVFIAALTRYKKLRNLTNLLIANLAISDFLVAIICCPFEMDYYVVRQLSWEHGHVLCASVNYLRTVSLYVSTNALLAIAIDRYLAIVHPLKPRMNYQTASFLIALVWMVSILIAIPSAYFATETVLFIVKSQEKIFCGQIWPVDQQLYYKSYFLFIFGVEFVGPVVTMTLCYARISRELWFKAVPGFQTEQIRKRLRCRRKTVLVLMCILTAYVLCWAPFYGFTIVRDFFPTVFVKEKHYLTAFYVVECIAMSNSMINTVCFVTVKNNTMKYFKKMMLLHWRPSQWGSKSSAELDLRTNGVPATEEVDCIRLK

SEQ ID NO: 3 - DNA, 1155 bp, Homo sapiens
atggcagcccagaatggaaacaccagtttcacacccaactttaatccaccccaagaccatgcctcctccctctcctttaacttcagttatggtgattatgacctccctatggatgaggatgaggacatgaccaagacccggaccttcttcgcagccaagatcgtcattggcattgcactggcaggcatcatgctggtctgcggcatcggtaactttgtctttatcgctgccctcacccgctataagaagttgcgcaacctcaccaatctgctcattgccaacctggccatctccgacttcctggtggccatcatctgctgccccttcgagatggactactacgtggtacggcagctctcctgggagcatggccacgtgctctgtgcctccgtcaactacctgcgcaccgtctccctctacgtctccaccaatgccttgctggccattgccattgacagatatctcgccatcgttcaccccttgaaaccacggatgaattatcaaacggcctccttcctgatcgccttggtctggatggtgtccattctcattgccatcccatcggcttactttgcaacagaaaccgtcctctttattgtcaagagccaggagaagatcttctgtggccagatctggcctgtggatcagcagctctactacaagtcctacttcctcttcatctttggtgtcgagttcgtgggccctgtggtcaccatgaccctgtgctatgccaggatctcccgggagctctggttcaaggcagtccctgggttccagacggagcagattcgcaagcggctgcgctgccgcaggaagacggtcctggtgctcatgtgcattctcacggcctatgtgctgtgctgggcacccttctacggtttcaccatcgttcgtgacttcttccccactgtgttcgtgaaggaaaagcactacctcactgccttctacgtggtcgagtgcatcgccatgagcaacagcatgatcaacaccgtgtgcttcgtgacggtcaagaacaacaccatgaagtacttcaagaagatgatgctgctgcactggcgtccctcccagcgggggagcaagtccagtgctgaccttgacctcagaaccaacggggtgcccaccacagaagaggtggactgtatcaggctgaagtga

SEQ ID NO: 4 - protein, 384 aa, Homo sapiens
MAAQNGNTSFTPNFNPPQDHASSLSFNFSYGDYDLPMDEDEDMTKTRTFFAAKIVIGIALAGIMLVCGIGNFVFIAALTRYKKLRNLTNLLIANLAISDFLVAIICCPFEMDYYVVRQLSWEHGHVLCASVNYLRTVSLYVSTNALLAIAIDRYLAIVHPLKPRMNYQTASFLIALVWMVSILIAIPSAYFATETVLFIVKSQEKIFCGQIWPVDQQLYYKSYFLFIFGVEFVGPVVTMTLCYARISRELWFKAVPGFQTEQIRKRLRCRRKTVLVLMCILTAYVLCWAPFYGFTIVRDFFPTVFVKEKHYLTAFYVVECIAMSNSMINTVCFVTVKNNTMKYFKKMMLLHWRPSQRGSKSSADLDLRTNGVPTTEEVDCIRLK

SEQ ID NO: 5 - DNA, 1149 bp, Homo sapiens
atggagaccaccatggggttcatggatgacaatgccaccaacacttccaccagcttcctttctgtgctcaaccctcatggagcccatgccacttccttcccattcaacttcagctacagcgactatgatatgcctttggatgaagatgaggatgtgaccaattccaggacgttctttgctgccaagattgtcattgggatggccctggtgggcatcatgctggtctgcggcattggaaacttcatctttatcgctgccctggtccgctacaagaaactgcgcaacctcaccaacctgctcatcgccaacctggccatctctgacttcctggtggccattgtctgctgcccctttgagatggactactatgtggtgcgccagctctcctgggagcacggccacgtcctgtgcacctctgtcaactacctgcgcactgtctctctctatgtctccaccaatgccctgctggccatcgccattgacaggtatctggctattgtccatccgctgagaccacggatgaagtgccaaacagccactggcctgattgccttggtgtggacggtgtccatcctgatcgccatcccttccgcctacttcaccaccgagacggtcctcgtcattgtcaagagccaggaaaagatcttctgcggccagatctggcctgtggaccagcagctctactacaagtcctacttcctctttatctttggcatagaattcgtgggccccgtggtcaccatgaccctgtgctatgccaggatctcccgggagctctggttcaaggcggtccctggattccagacagagcagatccgcaagaggctgcgctgccgcaggaagacggtcctggtgctcatgtgcatcctcaccgcctacgtgctatgctgggcgcccttctacggcttcaccatcgtgcgcgacttcttccccaccgtgtttgtgaaggagaagcactacctcactgccttctacatcgtcgagtgcatcgccatgagcaacagcatgatcaacactctgtgcttcgtgaccgtcaagaacgacaccgtcaagtacttcaaaaagatcatgttgctccactggaaggcttcttacaatggcgagtctcctgcaatgattcagacttggctttactggcttcttccatag

SEQ ID NO: 6 - protein, 382 aa, Homo sapiens
METTMGFMDDNATNTSTSFLSVLNPHGAHATSFPFNFSYSDYDMPLDEDEDVTNSRTFFAAKIVIGMALVGIMLVCGIGNFIFIAALVRYKKLRNLTNLLIANLAISDFLVAIVCCPFEMDYYVVRQLSWEHGHVLCTSVNYLRTVSLYVSTNALLAIAIDRYLAIVHPLRPRMKCQTATGLIALVWTVSILIAIPSAYFTTETVLVIVKSQEKIFCGQIWPVDQQLYYKSYFLFIFGIEFVGPVVTMTLCYARISRELWFKAVPGFQTEQIRKRLRCRRKTVLVLMCILTAYVLCWAPFYGFTIVRDFFPTVFVKEKHYLTAFYIVECIAMSNSMINTLCFVTVKNDTVKYFKKIMLLHWKASYNGESPAMIQTWLYWLLP

SEQ ID NO: 7 - DNA, 327 bp, Homo sapiens
atgaggagcctgtgctgcgccccactcctgctcctcttgctgctgccgccgctgctgctcacgccccgcgctggggatgccgccgtgatcaccggggcttgtgacaaggactcccaatgtggtggaggcatgtgctgtgctgtcagtatctgggtcaagagcataaggatttgcacacctatgggcaaactgggagacagctgccatccactgactcgtaaagttccattttttgggcggaggatgcatcacacttgcccatgtctgccaggcttggcctgtttacggacttcatttaaccgatttatttgtttagcccaaaagtaa

SEQ ID NO: 8 - protein, 118 aa, Homo sapiens
CAPLLLLLLLPPLLLPRAGDAAVITGACDKSQCGGGMCCAVSIWVSIRICTPMGKLGDSCPLTRKNNFGNGRQERRKRKRSKRKKEVPFFGRMHHTCPCLPGLACLTSFNRFICLAQK

SEQ ID NO: 9 - DNA, 1456 bp, Mus musculus
cgcgtccccaacgtcccgggtcccaacgccccggaacgcgtcccctaaccgccaccgcgtccccgggacgccatgggggacccgcgctgtgccccgctactgctacttctgctgctaccgctgctgttcacaccgcccgccggggatgccgcggtcatcaccggggcttgcgacaaggactctcagtgcggaggaggcatgtgctgtgctgtcagtatctgggttaagagcataaggatctgcacacctatgggccaagtgggcgacagctgccaccccctgactcggaaagttccattttgggggcggaggatgcaccacacctgcccctgcctgccaggcttggcgtgtttaaggacttctttcaaccggtttatttgcttggcccggaaatgatcactctgaagtaggaacttgaaatgcgaccctccgctgcacaatgtccgtcgagtctcacttgtaattgtggcaaacaaagaatactccagaaagaaatgttctcccccttccttgactttccaagtaacgtttctatctttgatttttgaagtggcttttttttttttttttttttcctttccttgaaggaaagttttgatttttggagagatttatagaggactttctgacatggcttctcatttccctgtttatgttttgccttgacatttttgaatgccaataacaactgttttcacaaataggagaataagagggaacaatctgttgcagaaacttccttttgccctttgccccactcgccccgccccgccccgccccgccctgcccatgcgcagacagacacacccttactcttcaaagactctgatgatcctcaccttactgtagcattgtgggtttctacacttccccgccttgctggtggacccactgaggaggctcagagagctagcactgtacaggtttgaaccagatcccccaagcagctcatttggggcagacgttgggagcgctccaggaactttcctgcacccatctggcccactggctttcagttctgctgtttaactggtgggaggacaaaattaacgggaccctgaaggaacctggcccgtttatctagatttgtttaagtaaaagacattttctccttgttgtggaatattacatgtctttttcttttttatctgaagctttttttttttctttaagtcttcttgttggagacattttaaagaacgccactcgaggaagcattgattttcatctggcatgacaggagtcatcattttaaaaaatcggtgttaagttataatttaaactttatttgtaacccaaaggtctaatgtaaatggatttcctgatatcctgccatttgtactggtatcaatatttctatgtaaaaaaaaaaaaaaattctgtatcagaataatgacaatactgtatatcctttgatttattttgatattatatccttatttttgtcaaaaaaaaa

SEQ ID NO: 10 - protein, 107 aa, Mus musculus
MGDPRCAPLLLLLLLPLLFTPPAGDAAVITGACDKDSQCGGGMCCAVSIWVKSIRICTPMGQVGDSCHPLTRKVPFWGRRMHHTCPCLPGLACLRTSFNRFICLARK

SEQ ID NO: 11 - DNA, 291 bp, Rana
atgaagtgttttgcacagattgtggtgttgctgcttgtaatagccttctcacatggtgctgttatcactggggcctgtgacaaagacgtacagtgcgggtcagggacctgctgcgctgccagtgcgtggtcacgtaacatcagattttgcatcccacttggaaacagcggggaggattgtcacccagccagtcataaggtgccttatgatggaaagcggttgagttccttgtgcccctgcaagtccggactaacttgctccaagtctggagaaaaatttaagtgttcttga

SEQ ID NO: 12 - protein, 96 aa, Rana
MKCFAQIVVLLLVIAFSHGAVITGACDKDVQCGSGTCCAASAWSRNIRFCIPLGNSGEDCHPASHKVPYDGKRLSSLCPCKSGLTCSKSGEKFKCS

SEQ ID NO: 13 - DNA, 390 bp, Homo sapiens
atgaggagcctgtgctgcgccccactcctgctcctcttgctgctgccgccgctgctgctcacgccccgcgctggggacgccgccgtgatcaccggggcttgtgacaaggactcccaatgtggtggaggcatgtgctgtgctgtcagtatctgggtcaagagcataaggatttgcacacctatgggcaaactgggagacagctgccatccactgactcgtaaaaacaattttggaaatggaaggcaggaaagaagaaagaggaagagaagcaaaaggaaaaaggaggttccattttttgggcggaggatgcatcacacttgcccatgtctgccaggcttggcctgtttacggacttcatttaaccgatttatttgtttagcccaaaagtaa

SEQ ID NO: 14 - protein, 129 aa, Homo sapiens
MRSLCCAPLLLLLLLPPLLLTPRAGDAAVITGACDKDSQCGGGMCCAVSIWVKSIRICTPMGKLGDSCHPLTRKNNFGNGRQERRKRKRSKRKKEVPFFGRRMHHTCPCLPGLACLRTSFNRFICLAQK

SEQ ID NO: 15 - DNA, 1415 bp, Homo sapiens
tggcctccccagcttgccaggcacaaggctgagcgggaggaagcgagaggcatctaagcaggcagtgttttgccttcaccccaagtgaccatgagaggtgccacgcgagtctcaatcatgctcctcctagtaactgtgtctgactgtgctgtgatcacaggggcctgtgagcgggatgtccagtgtggggcaggcacctgctgtgccatcagcctgtggcttcgagggctgcggatgtgcaccccgctggggcgggaaggcgaggagtgccaccccggcagccacaaggtccccttcttcaggaaacgcaagcaccacacctgtccttgcttgcccaacctgctgtgctccaggttcccggacggcaggtaccgctgctccatggacttgaagaacatcaatttttaggcgcttgcctggtctcaggatacccaccatccttttcctgagcacagcctggatttttatttctgccatgaaacccagctcccatgactctcccagtccctacactgactaccctgatctctcttgtctagtacgcacatatgcacacaggcagacatacctcccatcatgacatggtccccaggctggcctgaggatgtcacagcttgaggctgtggtgtgaaaggtggccagcctggttctcttccctgctcaggctgccagagaggtggtaaatggcagaaaggacattccccctcccctccccaggtgacctgctctctttcctgggccctgcccctctccccacatgtatccctcggtctgaattagacattcctgggcacaggctcttgggtgcattgctcagagtcccaggtcctggcctgaccctcaggcccttcacgtgaggtctgtgaggaccaatttgtgggtagttcatcttccctcgattggttaactccttagtttcagaccacagactcaagattggctcttcccagagggcagcagacagtcaccccaaggcaggtgtagggagcccagggaggccaatcagccccctgaagactctggtcccagtcagcctgtggcttgtggcctgtgacctgtgaccttctgccagaattgtcatgcctctgaggccccctcttaccacactttaccagttaaccactgaagcccccaattcccacagcttttccattaaaatgcaaatggtggtggttcaatctaatctgatattgacatattagaaggcaattagggtgtttccttaaacaactcctttccaaggatcagccctgagagcaggttggtgactttgaggagggcagtcctctgtccagattggggtgggagcaagggacagggagcagggcaggggctgaaaggggcactgattcagaccagggaggcaactacacaccaacatgctggctttagaataaaagcaccaactgaaaaaa

SEQ ID NO: 16 - protein, 105 aa, Homo sapiens
MRGATRVSIMLLLVTVSDCAVITGACERDVQCGAGTCCAISLWLRGLRMCTPLGREGEECHPGSHKVPFFRKRKHHTCPCLPNLLCSRFPDGRYRCSMDLKNINF

SEQ ID NO: 17 - DNA, 318 bp, Homo sapiens
atgagaggtgccacgcgagtctcaatcatgctcctcctagtaactgtgtctgactgtgctgtgatcacaggggcctgtgagcgggatgtccagtgtggggcaggcacctgctgtgccatcagcctgtggcttcgagggctgcggatgtgcaccccgctggggcgggaaggcgaggagtgccaccccggcagccacaagatccccttcttcaggaaacgcaagcaccacacctgtccttgcttgcccaacctgctgtgctccaggttcccggacggcaggtaccgctgctccatggacttgaagaacatcaatttttag

SEQ ID NO: 18 - protein, 105 aa, Homo sapiens [sequence omitted]

SEQ ID NO: 19 - protein, 81 aa, Artificial Sequence
  Mamba Intestinal Toxin
AVITGACERDLQCGKGTCCAVSLWIKSVRVCTPVGTSGEDCHPASHKIPFSGQRKMHHTCPCAPNLACVQTSPKKFKCLSK